(19) United States
(12) Patent Application Publication
(10) Pub. No.: US 2025/0235801 A1
(43) Pub. Date: Jul. 24, 2025

(54) KITS, ARTICLES, AND METHODS FOR BLOOD SEPARATION

(71) Applicant: Trustees of Tufts College, Medford, MA (US)

(72) Inventors: Charles R. Mace, Winchester, MA (US); Keith Baillargeon, Medford, MA (US)

(73) Assignee: Trustees of Tufts College, Medford, MA (US)

(21) Appl. No.: 18/703,626

(22) PCT Filed: Oct. 27, 2022
(86) PCT No.: PCT/US2022/048007

§ 371 Date: Apr. 22, 2024

Related U.S. Application Data
(60) Provisional application No. 63/273,727, filed on Oct. 29, 2021.

(51) Int. Cl. B01D 15/20

(52) U.S. Cl. CPC B01D 15/20

(57) ABSTRACT

Disclosed herein are kits, articles, and methods for blood separation, comprising: a support structure comprising an inlet, an outlet, and a channel between the inlet and the outlet; a separation device; and an absorbent layer; wherein the absorbent layer and support structure are configured such that the absorbent layer can be positioned in the support structure and be in fluidic connection with the inlet and the outlet of the support structure; wherein the separation device is removable from the support structure; wherein the outlet is a vessel and/or is configured to be in fluidic connection with a vessel.

### TECHNICAL FIELD

Kits, articles, and methods for blood separation are generally described.

### SUMMARY

Disclosed herein are kits, articles, and methods for blood separation. For example, inventive kits, articles, and methods that remove red blood cells from blood samples are described. In some embodiments, the kit comprises a support structure, an absorbent layer, a separation device (e.g., a removable separation device), a compression device, and/or a vessel. In some embodiments, the method comprises, in the support structure, passing a blood sample across the separation device to the absorbent layer, such that a blood sample with reduced number of red blood cells is collected inside the absorbent layer. The method may comprise removing the separation device from the support structure after the blood sample with reduced number of red blood cells has been passed into the absorbent layer. In some cases, the method comprises compressing the compression device against the absorbent layer after the separation device has been removed from the support structure. The method may comprise collecting the blood sample with reduced number of red blood cells in a vessel after compressing the compression device against the absorbent layer. In some embodiments, the kits, articles, and/or methods disclosed herein have one or more advantages, such as short separation time, short collection time, ease of separation (e.g., without constant manual operation or the use of a centrifuge), ease of collection (e.g., without the use of a centrifuge, vacuum, and/or any additional instruments), large loading capacity, large volume recovery, low amounts of clogging, low amounts of hemolysis in the recovered sample, high purity of the recovered sample, low amounts of mess (e.g., high containment of the blood within the article), low energy requirements, and/or ability to use whole blood samples without the need for dilution. The subject matter of the present invention involves, in some cases, interrelated products, alternative solutions to a particular problem, and/or a plurality of different uses of one or more systems and/or articles.

Some embodiments relate to kits.

In some embodiments, the kit comprises a support structure comprising an inlet, an outlet, and a channel between the inlet and the outlet; a separation device; an absorbent layer; and a compression device; wherein the absorbent layer and support structure are configured such that the absorbent layer can be positioned in the support structure and be in fluidic connection with the inlet and the outlet of the support structure; wherein the separation device is removable from the support structure; wherein the outlet is a vessel and/or is configured to be in fluidic connection with a vessel; and wherein the compression device and support structure are configured such that at least a portion of the compression device can be positioned at the inlet.

In some embodiments, the kit comprises a support structure comprising an inlet, an outlet, and a channel between the inlet and the outlet; and an absorbent layer; wherein the absorbent layer and support structure are configured such that the absorbent layer can be positioned in the support structure and be in fluidic connection with the inlet and the outlet of the support structure; wherein the outlet is a vessel and/or is configured to be in fluidic connection with a vessel; and wherein the absorbent layer has an absorbency of greater than or equal to 80 microliters/cm² and less than or equal to 600 microliters/cm².

In some embodiments, the kit comprises a support structure comprising an inlet, an outlet, and a channel between the inlet and the outlet; a separation device, wherein the separation device is removable from the support structure, and wherein the separation device comprises a first layer and a second layer; and an absorbent layer; wherein the absorbent layer and support structure are configured such that the absorbent layer can be positioned in the support structure and be in fluidic connection with the inlet and the outlet of the support structure; and wherein the outlet is a vessel and/or is configured to be in fluidic connection with a vessel.

In some embodiments, the kit comprises a support structure comprising an inlet, an outlet, and a channel connecting the inlet and the outlet; and an absorbent layer; wherein the absorbent layer and support structure are configured such that the absorbent layer can be positioned in the support structure and be in fluidic connection with the inlet and the outlet of the support structure; wherein the outlet is a vessel and/or is configured to be in fluidic connection with a vessel; and wherein the channel of the support structure has an internal volume of less than or equal to 10 milliliters.

Some embodiments relate to methods.

In some embodiments, the method comprises, in a support structure comprising an inlet, an outlet, a channel between the inlet and the outlet, a separation device positioned in the support structure, and an absorbent layer positioned in the support structure, performing the steps of: passing a blood sample across the separation device to the absorbent layer, such that a blood sample with reduced number of red blood cells is collected inside the absorbent layer; removing the separation device from the support structure after the blood sample with reduced number of red blood cells has been passed into the absorbent layer; and compressing a compression device against the absorbent layer after the separation device has been removed from the support structure.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is, in accordance with some embodiments, a cross-sectional schematic illustration of a kit comprising an absorbent layer positioned in a support structure (e.g., a hollow cylindrical support structure).

FIG. 1B is, in accordance with some embodiments, a cross-sectional schematic illustration of the kit of FIG. 1B when viewed from overhead.

FIG. 1C is, in accordance with some embodiments, a cross-sectional schematic illustration of a kit comprising an absorbent layer and a separation device positioned in a support structure (e.g., a hollow cylindrical support structure).

FIG. 1D is, in accordance with some embodiments, a cross-sectional schematic illustration of a kit comprising an absorbent layer and a compression device positioned in a support structure (e.g., a hollow cylindrical support structure).

FIG. 1E is, in accordance with some embodiments, a cross-sectional schematic illustration of a kit comprising an absorbent layer positioned in a support structure (e.g., a hollow cylindrical support structure) comprising an outlet in fluidic connection with a vessel.

FIG. 2A is, in accordance with some embodiments, a cross-sectional schematic illustration of a kit comprising an absorbent layer and a support structure.

FIG. 2B is, in accordance with some embodiments, a cross-sectional schematic illustration of a kit comprising an absorbent layer and a compression device.

FIG. 3 is, in accordance with some embodiments, a plot of the recovered volume of plasma versus the absorbent layer diameter.

FIG. 4 is, in accordance with some embodiments, a schematic illustration of an article comprising a first layer, a second layer, and a third layer.

FIG. 5 is a schematic of a deconstructed article, according to one set of embodiments.

FIG. 6 shows a method of separating blood, according to one set of embodiments.

FIG. 7 is a plot of the recovered plasma volume as a function of separation time, according to one set of embodiments. The large plasma separation device (1.6 cm diameter) was used. The sample input volume (250 μL) and hematocrit (ca. 45%) were constant. Each data point represents the average of three replicates and error bars represent the standard error of the mean.

FIG. 8 is a bar graph showing the separation efficiency of devices of various sizes with various sample input volumes, according to one set of embodiments. The separation time (10 mins) and hematocrit (ca. 45%) were constant. Each column represents the average (N=5) and error bars represent the standard error of the mean.

FIGS. 9A-9C show a comparison of plasma quality for samples prepared using plasma separation devices (N=20), in accordance with some embodiments, or a centrifuge (N=20).

FIG. 10A is a schematic of positive (test and control lines present) and negative (only control line present) results for a tetanus lateral flow test.

FIG. 10B shows images of a reference plasma sample collected via centrifugation of whole blood (positive control), a plasma sample recovered from a plasma separation device in accordance with some embodiments (collected plasma), a plasma sample recovered from a plasma separation device in accordance with some embodiments after drying at room temperature for 16 hours and elution with buffer (rehydrated plasma), and a buffered sample without tetanus antibody (negative control).

FIG. 10C shows replicate images of lateral flow tests with plasma samples recovered from a plasma separation device in accordance with some embodiments and directly applied to the lateral flow test without centrifugation (N=5).

FIG. 11 shows the dimensions for various acrylic scaffolds, according to one set of embodiments.

FIGS. 12A-12B show the quantitation of total protein, where FIG. 12A shows the calibration curve used and FIG. 12B shows the replicate data for plasma obtained from a device in accordance with some embodiments compared to the plasma obtained from centrifugation (N=20, p-value=0.0001).

FIGS. 13A-B show the calibration data for purity assessment, where FIG. 13A is a plot of four calibration curves used for h-IgG, and FIG. 13B shows the calibration plot for hemoglobin.

### DETAILED DESCRIPTION

Disclosed herein are kits, articles, and methods for blood separation. For example, inventive kits, articles, and methods that remove red blood cells from blood samples are described. In some embodiments, blood separation (e.g., removal of red blood cells from a blood sample) is desired, as removal of the cellular components (e.g., red and white blood cells) from whole blood can improve sensitivity of some clinical assays and/or reduce degradation of analytes of interest in plasma. However, this separation can be challenging, as the red blood cells in whole blood are numerous and may clog separation devices, and red blood cells are fragile and may rupture, contaminating the plasma. Moreover, this separation can be expensive, as it may require expensive high-speed centrifuges or constant manual operation, and it may produce only low volumes of plasma for large separation devices and/or long separation times. In some embodiments, the articles and/or methods described herein provide improved articles and/or methods for blood separation.

In some embodiments, the kit comprises a support structure, an absorbent layer, a separation device (e.g., a removable separation device), a compression device, and/or a vessel. In some cases, the method comprises, in the support structure, passing a blood sample across the separation device to the absorbent layer, such that a blood sample with reduced number of red blood cells is collected inside the absorbent layer. The method may involve removing the separation device from the support structure after the blood sample with reduced number of red blood cells has been passed into the absorbent layer. In some instances, the method comprises compressing the compression device against the absorbent layer after the separation device has been removed from the support structure. The method may involve collecting the blood sample with reduced number of red blood cells in a vessel after compressing the compression device against the absorbent layer.

In some embodiments, the kits, articles, and/or methods disclosed herein have one or more advantages, such as short separation time, short collection time, ease of separation (e.g., without constant manual operation or the use of a centrifuge), ease of collection (e.g., without the use of a centrifuge, vacuum, and/or any additional instruments), large loading capacity, large volume recovery, low amounts of clogging, low amounts of hemolysis in the recovered sample, high purity of the recovered sample, low amounts of mess (e.g., high containment of the blood within the article), low energy requirements, and/or ability to use whole blood samples without the need for dilution.

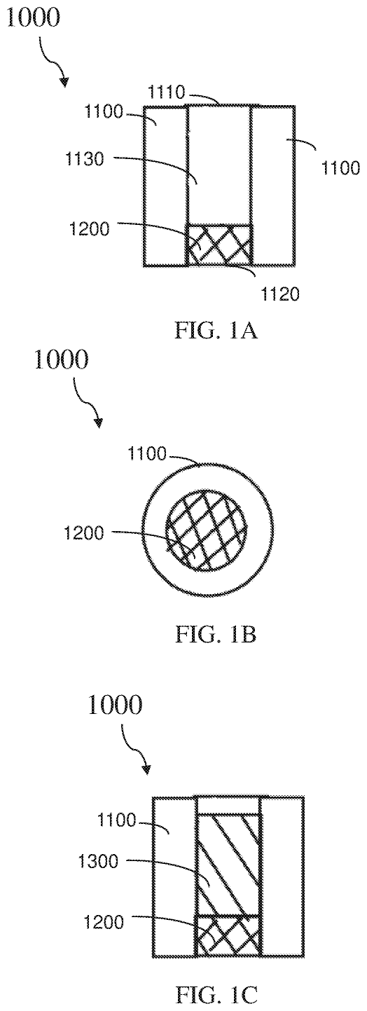

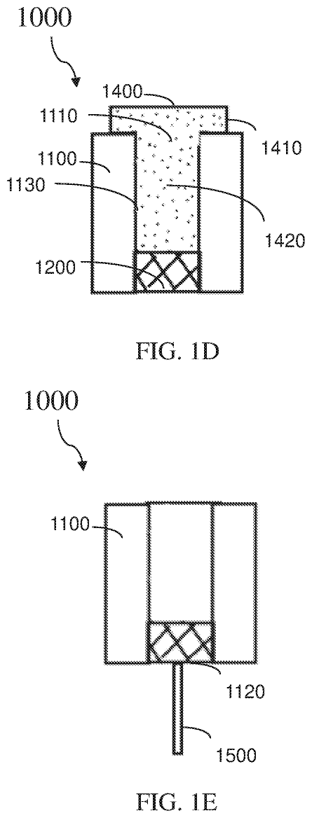

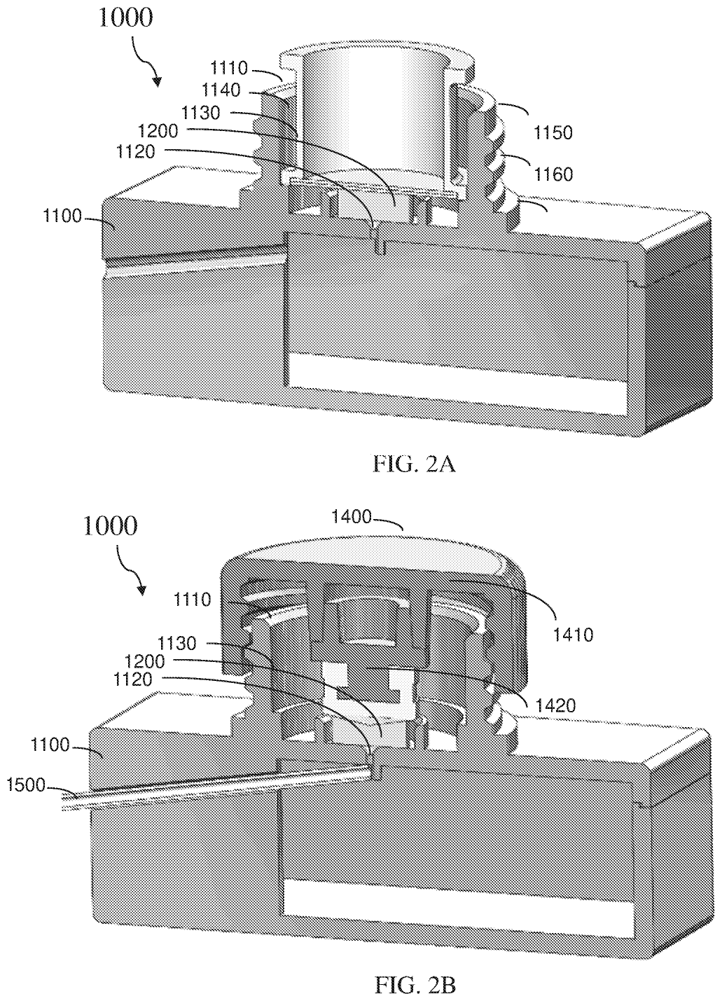

Kits are described herein. In accordance with some embodiments, kits are illustrated schematically in FIGS. 1A-2B. According to some embodiments, the kit comprises any article or component disclosed herein, or combinations thereof.

In some embodiments, the kit comprises a support structure (e.g., any support structure disclosed herein). The support structure may be used for supporting, holding and/or containing one or more components such as an absorbent layer, a separation device (e.g., a removable separation device), and/or a compression device as described herein. For example, in FIGS. 1A-1B, in some cases, a kit 1000 comprises a support structure 1100. In FIGS. 1A-1B, support structure 1100 is shown as a hollow cylinder, although it should be understood that support structure 1100 can have other shapes and forms, in some instances. According to some embodiments, the support structure comprises an inlet, an outlet, and/or a channel between the inlet and the outlet. For example, in FIG. 1A, in accordance with some embodiments, support structure 1100 comprises an inlet 1110, an outlet 1120, and a channel 1130 between inlet 1110 and outlet 1120.

In some embodiments, the outlet is a vessel and/or is configured to be in fluidic connection (and/or is in fluidic connection) with a vessel. The vessel may be used for containing a fluid such as a fluid sample received from the support structure. For example, in FIG. 1A, in accordance with some embodiments, outlet 1120 is configured to be in fluidic connection with a vessel. As another example, in FIG. 1E, in some instances, outlet 1120 is in fluidic connection with a vessel 1500. As used herein, two components are in fluidic connection when fluid can pass from one component to the other component (e.g., in one direction only or in both directions).

Non-limiting examples of suitable vessels include a capillary tube, a cuvette, a test tube, a beaker, a flask, and/or a conical tube. In some cases, the vessel is disposable. In some instances, the vessel is reusable. According to some embodiments, the vessel comprises glass and/or plastic. In some embodiments, the vessel is transparent and/or comprises a transparent portion.

The vessel may have any suitable internal volume. For example, the vessel may have an internal volume of less than or equal to 10 milliliters, less than or equal to 8 milliliters, less than or equal to 6 milliliters, less than or equal to 4 milliliters, less than or equal to 2 milliliters, less than or equal to 1 milliliter, less than or equal to 750 microliters, less than or equal to 500 microliters, less than or equal to 400 microliters, less than or equal to 300 microliters, less than or equal to 200 microliters, or less than or equal to 100 microliters. In some embodiments, the vessel has an internal volume of greater than or equal to 1 microliter, greater than or equal to 5 microliters, greater than or equal to 10 microliters, greater than or equal to 25 microliters, greater than or equal to 50 microliters, greater than or equal to 100 microliters, greater than or equal to 150 microliters, greater than or equal to 200 microliters, greater than or equal to 250 microliters, greater than or equal to 500 microliters, greater than or equal to 750 microliters, greater than or equal to 1 milliliter, greater than or equal to 2 milliliters, or greater than or equal to 3 milliliters. Combinations of these ranges are also possible (e.g., greater than or equal to 1 microliter and less than or equal to 10 milliliters, greater than or equal to 1 microliter and less than or equal to 500 microliters, or greater than or equal to 10 microliters and less than or equal to 200 microliters).

The channel, support structure, or portion thereof (e.g., inlet, outlet, and channel combined) may have any suitable internal volume. For example, in some cases, the channel, support structure, or portion thereof (e.g., inlet, outlet, and channel combined) has an internal volume of less than or equal to 10 milliliters, less than or equal to 8 milliliters, less than or equal to 6 milliliters, less than or equal to 4 milliliters, less than or equal to 2 milliliters, less than or equal to 1 milliliter, less than or equal to 750 microliters, or less than or equal to 500 microliters. In some instances, the channel, support structure, or portion thereof (e.g., inlet, outlet, and channel combined) has an internal volume of greater than or equal to 50 microliters, greater than or equal to 100 microliters, greater than or equal to 150 microliters, greater than or equal to 200 microliters, greater than or equal to 250 microliters, greater than or equal to 500 microliters, greater than or equal to 750 microliters, greater than or equal to 1 milliliter, greater than or equal to 2 milliliters, or greater than or equal to 3 milliliters. Combinations of these ranges are also possible (e.g., greater than or equal to 50 microliters and less than or equal to 10 milliliters). As used herein, the internal volume of a component is the total volume of fluid that could be contained within that component at one time.

The channel, support structure, or portion thereof (e.g., inlet, outlet, and channel combined) may have any suitable height. For example, in some cases, the channel, support structure, or portion thereof (e.g., inlet, outlet, and channel combined) has a height greater than the thickness of the separation device combined with the thickness of the absorbent layer. In some instances, the channel, support structure, or portion thereof (e.g., inlet, outlet, and channel combined) has a height less than or equal to the length of the compression device or a portion thereof (e.g., a plunger portion of the compression device) combined with the thickness of the absorbent layer.

In some embodiments, the channel, support structure, or portion thereof (e.g., inlet, outlet, and channel combined) has a height of less than or equal to 20 centimeters, less than or equal to 18 centimeters, less than or equal to 16 centimeters, less than or equal to 14 centimeters, less than or equal to 12 centimeters, less than or equal to 10 centimeters, less than or equal to 8 centimeters, less than or equal to 6 centimeters, less than or equal to 4 centimeters, less than or equal to 2 centimeters, less than or equal to 1 centimeter, less than or equal to 8 millimeters, less than or equal to 6 millimeters, less than or equal to 4 millimeters, or less than or equal to 2 millimeters. In some embodiments, the channel, support structure, or portion thereof (e.g., inlet, outlet, and channel combined) has a height of greater than or equal to 0.01 millimeters, greater than or equal to 0.05 millimeters, greater than or equal to 0.1 millimeters, greater than or equal to 0.3 millimeters, greater than or equal to 0.5 millimeters, greater than or equal to 0.7 millimeters, greater than or equal to 1 millimeter, greater than or equal to 2 millimeters, greater than or equal to 4 millimeters, greater than or equal to 6 millimeters, greater than or equal to 8 millimeters, greater than or equal to 1 centimeter, greater than or equal to 2 centimeters, greater than or equal to 4 centimeters, greater than or equal to 6 centimeters, greater than or equal to 8 centimeters, greater than or equal to 10 centimeters, greater than or equal to 12 centimeters, or greater than or equal to 14 centimeters. Combinations of these ranges are also possible (e.g., greater than or equal to 0.01 millimeters and less than or equal to 20 centimeters, greater than or equal to 0.05 millimeters and less than or equal to 10 centimeters, greater than or equal to 0.1 millimeters and less than or equal to 6 centimeters, or greater than or equal to 1 centimeter and less than or equal to 6 centimeters).

In some embodiments, the kit comprises an absorbent layer (e.g., any absorbent layer and/or third layer disclosed herein). For example, in FIGS. 1A-1B, in some cases, kit 1000 comprises an absorbent layer 1200.

According to some embodiments, the absorbent layer and support structure are configured such that the absorbent layer can be positioned in the support structure (e.g., in the channel) and be in fluidic connection with the inlet and the outlet of the support structure and/or the absorbent layer is positioned in the support structure (e.g., in the channel, e.g., in fluidic connection with the inlet and the outlet of the support structure). For example, in FIGS. 1A-1B, in some instances, absorbent layer 1200 is positioned in support structure 1100 such that it is in fluidic connection with inlet 1110 and outlet 1120. It should be understood that although FIGS. 1A-1B show absorbent layer 1200 positioned in support structure 1100, the kit includes the absorbent layer outside of the support structure (e.g., configured such that the absorbent layer can be positioned in the support structure), in some cases.

In some embodiments, the support structure is configured to be used with separation devices of different sizes (e.g., different maximum horizontal dimensions) and/or absorbent layers of different sizes (e.g., different maximum horizontal dimensions). In some embodiments, a maximum horizontal dimension of the absorbent layer and/or separation device (or one or more layers thereof) may be selected based on the desired sample (e.g., blood sample) input volume and/or the desired volume of sample recovered (e.g., volume of plasma recovered and/or volume of the blood sample with reduced number of red blood cells passed into the absorbent layer and/or collected in the vessel).

In accordance with some embodiments, the absorbent layer is secured to the support structure. In some cases, the absorbent layer is secured to the support structure using adhesive (e.g., any adhesive disclosed herein, such as a UV cured adhesive). In some instances, the absorbent layer is secured to the support structure due to its positioning between ridges (e.g., horizontal ridges and/or vertical ridges) in the support structure. Other configurations for supporting the absorbent layer are also possible.

In some embodiments, the kit comprises a separation device (e.g., any separation device or article disclosed herein, or portion thereof, such as an article disclosed herein without the third layer). For example, in FIG. 1C, in accordance with some embodiments, kit 1000 comprises a separation device 1300. In some cases, the separation device and support structure are configured such that the separation device can be positioned in the support structure (e.g., in fluidic connection with the inlet and the outlet of the support structure) (e.g., in the channel) and/or the separation device is positioned in the support structure (e.g., in the channel) (e.g., in fluidic connection with the inlet and the outlet of the support structure). It should be understood that although FIG. 1C shows separation device 1300 positioned in support structure 1100, the kit includes the separation device outside of the support structure (e.g., configured such that the separation device can be positioned in the support structure upon use), in some instances.

In some embodiments, the separation devices comprises a first layer (e.g., any first layer disclosed herein) and a second layer (e.g., any second layer disclosed herein). According to some embodiments, the separation device is configured to be removable from the support structure. In other words, the separation device is not integrally connected to the support structure. Two or more objects are integrally connected when the objects do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.). Here, the separation device is not integrally connected to the support structure in some embodiments; for example, in FIG. 1C, separation device 1300 may be removable from support structure 1100 during the course of normal use.

According to some embodiments, the kit comprises a compression device. For example, in FIG. 1C, in some instances, kit 1000 comprises a compression device 1400. The compression device may have a shape, volume and/or size that mates with and/or is complementary to at least a portion of the support structure. In some cases, the compression device and support structure are configured such that at least a portion of the compression device can be positioned inside (e.g., inside a cavity of) at least a portion of the support structure, such as at the inlet (e.g., at the entrance of the inlet and/or partially or fully in the inlet and/or channel) of the support structure. For example, in FIG. 1C, in accordance with some embodiments, compression device 1400 and support structure 1100 are configured such that at least a portion of compression device 1400 can be positioned at inlet 1110 (e.g., in inlet 1110 and, optionally, in channel 1130).

In some embodiments, the compression device is removable from the support structure. In other embodiments, the compression device is not removable from the support structure after it is compressed against the absorbent layer. For instance, in some such embodiments the compression device may be integrally connected to the support structure (e.g., before and/or after it is compressed against the absorbent layer).

In some embodiments, the compression device comprises a cap and/or a plunger. For example, in FIG. 1D, in accordance with some embodiments, compression device 1400 comprises cap 1410 and plunger 1420. In some cases, the plunger is configured to compress the absorbent layer (e.g., when the compression device is placed at the inlet (e.g., at the entrance of the inlet and/or partially or fully in the inlet and/or channel of the support structure) of the support structure.

In some instances, the kit comprises a cap that is separate from the compression device (e.g., comprising a plunger). In some embodiments, the cap is attached to (e.g., with a hinge and/or tether) and/or is part of the support structure. For instance, in some such embodiments the cap may be integrally connected to the support structure. According to some embodiments, the cap (e.g., the cap portion of the compression device and/or the cap that is separate from the compression device) is configured to seal the inlet of the support structure such that liquid (e.g., blood and/or water) cannot be transported from the absorbent layer through the inlet to an exterior of the support structure and/or liquid cannot be transported from an exterior of the support structure through the inlet to the absorbent layer. Sealing the inlet may prevent contamination of the sample and/or may protect the user from the sample (e.g., a biological hazard).

In some embodiments, the compression device comprises one or more ridges. For example, the compression device may comprise greater than or equal to 1 ridge, greater than or equal to 2 ridges, greater than or equal to 3 ridges, or greater than or equal to 4 ridges. In some embodiments, the compression device comprises less than or equal to 10 ridges, less than or equal to 8 ridges, less than or equal to 6 ridges, less than or equal to 5 ridges, less than or equal to 4 ridges, or less than or equal to 3 ridges. Combinations of these ranges are also possible (e.g., greater than or equal to 1 and less than or equal to 10 ridges or greater than or equal to 1 and less than or equal to 4 ridges). In some cases, the compression device comprises one ridge. For example, in some instances, the compression device comprises one ridge that winds down the compression device or a portion thereof (e.g., like the ridges of a screw). According to some embodiments, one or more of the one or more ridges (e.g., all of the ridges) are on an interior surface of the compression device. In some embodiments, one or more of the one or more ridges (e.g., all of the ridges) are on an exterior surface of the compression device.

In some embodiments, the support structure (e.g., the inlet and/or channel) comprises an interior surface and an exterior surface. For example, in FIG. 2A, in accordance with some embodiments, support structure 1100 comprises interior surface 1140 (e.g., of the inlet and/or channel) and exterior surface 1150 (e.g., of the inlet and/or channel). In some cases, the support structure (e.g., the inlet and/or channel) comprises one or more ridges. For example, in FIG. 2A, in accordance with some embodiments, exterior surface 1150 comprises ridge 1160. In some instances, the one or more ridges (e.g., of the exterior surface) are configured to secure the compression device to the support structure. In some such embodiments, one or more ridges of the compression device (e.g., on an interior surface of the compression device) are configured to mate with and/or secure to one or more ridges of the support structure (e.g., the exterior of the support structure, such as the exterior of the inlet and/or channel of the support structure).

For example, in some embodiments, the compression device and/or support structure are configured such that when the compression device is compressed onto the support structure, one or more ridges of the compression device is pushed past one or more ridges of the support structure, such that the one or more ridges of the support structure secures the compression device in place. In another example, in some instances, the compression device is configured to screw onto the support structure. For example, in some cases, the compression device is configured to screw onto the support structure via the one or more ridges of the exterior surface of the support structure (e.g., the exterior surface of the inlet and/or channel) (e.g., the one or more ridges of the compression device (e.g., on an interior surface of the compression device) are configured to screw onto the one or more ridges of the support structure).

According to some embodiments, one or more components of the kit (e.g., the support structure and/or the compression device, or a portion thereof) are 3D printed and/or injection molded.

In some cases, the kit and/or one or more components thereof is disposable. In some instances, the kit and/or one or more components thereof is (e.g., all of the components of the kit) are reusable (e.g., after washing and/or sterilizing). According to some embodiments, one or more components of the kit (e.g., the absorbent layer, the separation device, and/or the vessel) are disposable while one or more components of the kit (e.g., the support structure, the compression device, and/or the vessel) are reusable (e.g., after washing and/or sterilizing). In some such embodiments, the kit comprises multiple (e.g., greater than or equal to 2, greater than or equal to 3, or greater than or equal to 4; less than or equal to 10, less than or equal to 8, or less than or equal to 5; combinations are also possible) of the disposable components (e.g., multiple absorbent layers, multiple separation devices, and/or multiple vessels).

In some embodiments, the kit and/or one or more components thereof is sterile. In some cases, the kit and/or one or more components thereof are, together or individually, packaged. In some instances, the packaging maintains sterility.

According to some embodiments, the kit is configured to separate a blood sample to produce a blood sample with reduced number of red blood cells and/or to collect the blood sample with reduced number of red blood cells (e.g., in the absorbent layer). In accordance with some embodiments, the kit is configured to passively (e.g., without the use of a centrifuge and/or without any force besides gravity) separate a blood sample to produce a blood sample with reduced number of red blood cells and/or to collect the blood sample with reduced number of red blood cells (e.g., in the absorbent layer).

In some embodiments, the kit comprises multiple (e.g., greater than or equal to 1, greater than or equal to 2, or greater than or equal to 3; less than or equal to 5, less than or equal to 4, or less than or equal to 3; combinations of these ranges are also possible) absorbent layers with different maximum horizontal dimensions (e.g., differing by greater than or equal to 0.1 mm, greater than or equal to 0.5 mm, greater than or equal to 1 mm, greater than or equal to 2 mm, or greater than or equal to 3 mm; and/or less than or equal to 10 mm, less than or equal to 7 mm, less than or equal to 5 mm, or less than or equal to 3 mm; combination of these ranges are also possible). In some embodiments, the kit comprises multiple separation devices (e.g., greater than or equal to 1, greater than or equal to 2, or greater than or equal to 3; and/or less than or equal to 5, less than or equal to 4, or less than or equal to 3; combinations of these ranges are also possible) with different maximum horizontal dimensions (e.g., differing by greater than or equal to 0.1 mm, greater than or equal to 0.5 mm, greater than or equal to 1 mm, greater than or equal to 2 mm, or greater than or equal to 3 mm; less than or equal to 10 mm, less than or equal to 7 mm, less than or equal to 5 mm, or less than or equal to 3 mm; combination of these ranges are also possible). For example, in a case where the separation devices in the kit each comprise one or more layers, in some embodiments, a first separation device within the kit has a different maximum horizontal dimension for at least one of its layers (e.g., all of its layers) compared to the maximum horizontal dimension for at least one of the layers (e.g., all of the layers) of a second separation device within the kit.

In some embodiments, the kit comprises instructions. The instructions may recite one or more method steps disclosed herein.

Methods are described herein. In accordance with some embodiments, the methods can be understood in view of FIGS. 1A-2B. In some embodiments, the method comprises a method of using any kit, article, or component thereof disclosed herein, or combinations thereof. For example, in some cases, the method comprises performing steps in a kit, article, or component thereof disclosed herein, such as in a support structure comprising an inlet, an outlet, a channel between the inlet and the outlet, a separation device positioned in the support structure, and an absorbent layer positioned in the support structure.

In some instances, the method comprises passing a sample (e.g., any sample disclosed herein, such as a blood sample) across the separation device to the absorbent layer. For example, in accordance with some embodiments, the method comprises passing the sample (e.g., blood sample) across separation device 1300 to absorbent layer 1200 in FIG. 1C. According to some embodiments, the method comprises passing the blood sample across the separation device to the absorbent layer such that a blood sample with reduced number of red blood cells is collected inside the absorbent layer. It should be understood that any disclosure herein for a sample with reduced number of red blood cells, reduced red blood cells, or further reduced red blood cells applies to each, in some cases.

In some instances, the method comprises removing the separation device from the support structure. For example, in accordance with some embodiments, the method comprises removing separation device 1300 from support structure 1100 in FIG. 1C. In some embodiments, the method comprises removing the separation device from the support structure after the blood sample with reduced number of red blood cells has been passed into the absorbent layer.

In some embodiments, the method comprises compressing a compression device against the absorbent layer. For example, in accordance with some embodiments, the method comprises compressing compression device 1400 against absorbent layer 1200 in FIG. 1D. In some cases, the method comprises compressing a compression device against the absorbent layer after the separation device has been removed from the support structure.

In some instances, the method comprises collecting the blood sample with reduced number of red blood cells in a vessel. For example, in FIG. 1E, in accordance with some embodiments, the method comprises collecting the blood sample with reduced number of red blood cells in vessel 1500. In some embodiments, the method comprises collecting the blood sample with reduced number of red blood cells in a vessel after compressing the compression device against the absorbent layer.

In some embodiments, the method comprises passively (e.g., without the use of a centrifuge and/or without any force besides gravity) separating a blood sample to produce a blood sample with reduced number of red blood cells and/or collecting the blood sample with reduced number of red blood cells (e.g., in the absorbent layer).

In some embodiments, the method comprises selecting a maximum horizontal dimension of an absorbent layer and/or a maximum horizontal dimension of a separation device (or one or more layers thereof) based on the desired sample input volume and/or the desired volume of sample recovered (e.g., volume of plasma recovered and/or volume of the blood sample with reduced number of red blood cells passed into the absorbent layer and/or collected in the vessel). For example, in some cases, if a smaller sample input volume is desired, a smaller maximum horizontal dimension of one or more layers (e.g., an absorbent layer and/or a separation device or one or more layers thereof) may be selected than if a larger sample input volume were desired, as this would result in increased volume of sample recovered and/or increased separation efficiency, in some instances. As another example, in some cases, if a larger volume of sample recovered is desired, a larger maximum horizontal dimension (and, optionally, a larger sample input volume) of one or more layers (e.g., an absorbent layer and/or a separation device or one or more layers thereof) may be selected than if a smaller volume of sample recovered were desired, as this would result in increased volume of sample recovered and/or increased separation efficiency, in some instances.

Certain embodiments relate to articles. Examples of suitable articles are disclosed in International Patent Application Number PCT/US2021/015624, filed Jan. 29, 2021 and published as WO 2021/155096, which is hereby incorporated by reference in its entirety. In accordance with some embodiments, articles are illustrated schematically in FIGS. 1-2. According to some embodiments, the kit comprises an article disclosed herein or a portion thereof.

In some embodiments, the absorbent layer is part of an article (e.g., the third layer of an article). In some embodiments, the article comprises multiple layers (e.g., a first layer and a second layer). In some embodiments, the separation device is part of an article (e.g., as a combination of a first layer and a second layer) (e.g., the same article comprising the absorbent layer). In some embodiments, the article comprises a first layer, a second layer, and a third layer (e.g., any absorbent layer disclosed herein). It should be understood that any description related to the article may apply to any of the layers individually (e.g., the third layer individually), or any combination of the layers (e.g., the first and second layers together, or all three layers together). Similarly, it should be understood that any description related to the layers within the article and/or when combined with other layers may apply to the layer individually (e.g., any description herein related to the third layer may apply to the third layer individually or the third layer when combined with other layers, regardless of the context in which it is described). It should also be appreciated that not all layers shown in the figures and described herein need be present in all embodiments. For instance, in some cases the first and/or second layer(s) is/are optional, and an article may include only the third layer, a combination of the third layer with the first layer, or a combination of the third layer and the second layer. Other configurations are also possible.

In some embodiments, the first layer is a pre-filter layer that quickly removes a significant portion of the red blood cells (and/or white blood cells) from whole blood, such that the second layer is less likely to get clogged and/or can have a higher loading capacity. In some embodiments, the second layer further removes red blood cells (and/or white blood cells). In some embodiments, the second layer has a gradient in pore size (e.g., with larger pores on the surface of the second layer adjacent to the first layer), such that the second layer is less likely to get clogged and/or is less likely to rupture the red blood cells. In some embodiments, the third layer is absorbent, so that it can absorb the purified blood. In some embodiments, the purified blood in the third layer can be used immediately (e.g., collected from and/or used directly from the third layer) or it can be stored long term (e.g., dried in the third layer). In some embodiments, the first layer, second layer, and third layer (e.g., absorbent layer) are vertically stacked (e.g., in the support structure, such as in the inlet and/or channel).

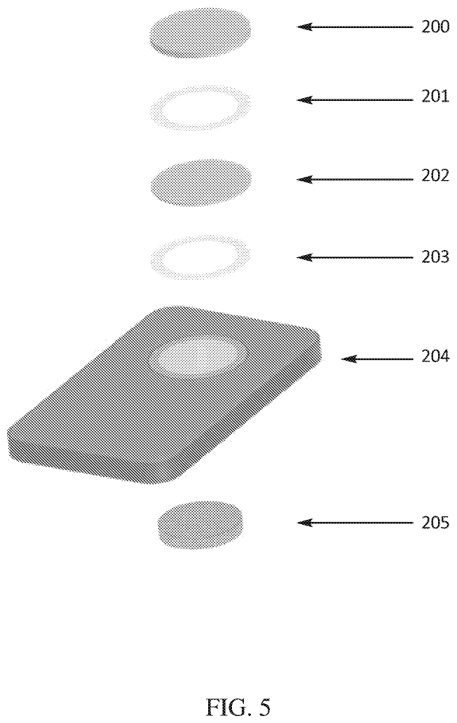

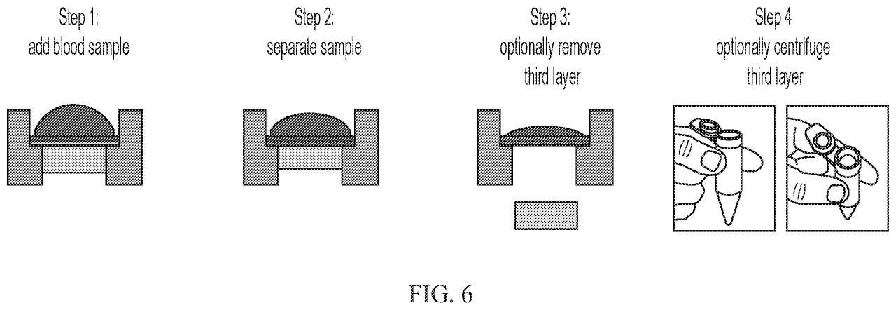

In some embodiments, the article comprises one or more layers. In some embodiments, the article comprises greater than or equal to 1 layer, greater than or equal to 2 layers, or greater than or equal to 3 layers. In some embodiments, the article comprises less than or equal to 10 layers, less than or equal to 7 layers, less than or equal to 5 layers, less than or equal to 4 layers, or less than or equal to 3 layers. Combinations of these ranges are also possible (e.g., greater than or equal to 1 layer and less than or equal to 4 layers). In some embodiments, the article comprises a first layer, a second layer, and a third layer. For example, in some embodiments, article 100 in FIG. 4 comprises first layer 110, second layer 120, and third layer 130. Similarly, in some embodiments, the article in FIG. 5 comprises first layer 200, second layer 202, and third layer 205.

In some embodiments, the article comprises a first layer. In some embodiments, the first layer comprises a pre-filter. In some embodiments, the first layer comprises fiberglass, polyester, a fibrous membrane (e.g., polyether sulfone), and/or mesh (e.g., polyester and/or nylon). In some embodiments, the polyester comprises a treated polyester, such as Leukosorb. In some embodiments, the first layer comprises a mesh (e.g., polyester and/or nylon). In some embodiments, the first layer is treated. In some embodiments, the first layer is not treated. The first layer may be fibrous or non-fibrous.

In some embodiments, the first layer is porous. In some embodiments, the first layer has a first mode pore size. In some embodiments, the first mode pore size is greater than or equal to 1 micron, greater than or equal to 2 microns, greater than or equal to 3 microns, greater than or equal to 4 microns, greater than or equal to 5 microns, greater than or equal to 10 microns, or greater than or equal to 15 microns. In some embodiments, the first mode pore size is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 1 micron and less than or equal to 30 microns, greater than or equal to 1 micron and less than or equal to 6 microns, greater than or equal to 2 microns and less than or equal to 25 microns, or greater than or equal to 15 microns and less than or equal to 25 microns).

In some embodiments, the first layer can have a variety of suitable thicknesses. In some embodiments, the first layer has a relatively small thickness. In some embodiments, the thickness of the first layer is greater than or equal to 150 microns, greater than or equal to 165 microns, or greater than or equal to 180 microns. In some embodiments, the thickness of the first layer is less than or equal to 500 microns, less than or equal to 400 microns, less than or equal to 300 microns, less than or equal to 250 microns, or less than or equal to 220 microns. Combinations of these ranges are also possible (e.g., greater than or equal to 180 microns and less than or equal to 220 microns, or greater than or equal to 150 microns and less than or equal to 500 microns). In some embodiments, the relatively small thickness of the first layer reduces separation time.

In some embodiments, the first layer has a relatively low absorbency. In some embodiments, the absorbency of the first layer is less than or equal to 100 microliters/cm², less than or equal to 90 microliters/cm², less than or equal to 80 microliters/cm², less than or equal to 70 microliters/cm², less than or equal to 60 microliters/cm², less than or equal to 50 microliters/cm², less than or equal to 40 microliters/cm², less than or equal to 30 microliters/cm², less than or equal to 20 microliters/cm², less than or equal to 15 microliters/cm², less than or equal to 10 microliters/cm², or less than or equal to 5 microliters/cm². In some embodiments, the absorbency of the first layer is greater than or equal to 10 microliters/cm², greater than or equal to 15 microliters/cm², greater than or equal to 20 microliters/cm², greater than or equal to 30 microliters/cm², or greater than or equal to 40 microliters/cm². Combinations of these ranges are also possible (e.g., greater than or equal to 10 microliters/cm² and less than or equal to 100 microliters/cm² or greater than or equal to 20 microliters/cm² and less than or equal to 50 microliters/cm²). In some embodiments, the relatively low absorbency of the first layer increases the separation efficiency and/or the volume of sample recovered (e.g., increases the yield of the separation), as a lower volume of the blood plasma may be retained by the first layer.

In some embodiments, the first layer comprises multiple sub-layers. For example, in some embodiments, the first layer has greater than or equal to 2 sub-layers, greater than or equal to 3 sub-layers, or greater than or equal to 4 sub-layers. In some embodiments, the first layer has less than or equal to 10 sub-layers, less than or equal to 7 sub-layers, less than or equal to 5 sub-layers, less than or equal to 4 sub-layers, less than or equal to 3 sub-layers, or less than or equal to 2 sub-layers. Combinations of these ranges are also possible (e.g., greater than or equal to 2 sub-layers and less than or equal to 10 sub-layers, or greater than or equal to 2 sub-layers and less than or equal to 4 sub-layers). In embodiments where the first layer comprises multiple sub-layers, the sub-layers may each independently have any features described herein for the first layer.

In embodiments where the first layer comprises multiple sub-layers, multiple of the sub-layers (e.g., all of the sub-layers) may comprise the same material or different material. For example, in some embodiments, the first layer comprises three sub-layers, and all of the sub-layers comprise a mesh (e.g., a polyester and/or nylon mesh). In some embodiments, one or more properties (e.g., thickness, mode pore size, mean pore size, maximum horizontal dimension, and/or absorbency) of the sub-layers (e.g., all of the sub-layers) are the same or different. In some embodiments where each of the sub-layers have a different property (e.g., mode pore size), the sub-layers are arranged such that a gradient in that property is formed. As a non-limiting example, in some embodiments, the first layer comprises three sub-layers, and each of the sub-layers has a different mode pore size such that a gradient in mode pore size is formed (e.g., 11 micron mode pore size in the first sub-layer, 6 micron mode pore size in the second sub-layer, and 1 micron mode pore size in the third sub-layer, wherein the second sub-layer is positioned between the first sub-layer and the third sub-layer).

In some embodiments, the article comprises a second layer. In some embodiments, the second layer comprises a polymer. In some embodiments, the second layer comprises polyether sulfone. In some embodiments, the second layer comprises a plasma separation membrane, such as a Pall plasma separation membrane (e.g., a Pall Vivid plasma separation membrane (e.g., grade GX, GR, and/or GF)), a Kinbio plasma separation membrane, and/or a Cobetter plasma separation membrane. The second layer may be fibrous or non-fibrous.

In some embodiments, the second layer is porous. In some embodiments, the second layer has a second mode pore size. In some embodiments, the second mode pore size (the mode pore size of the second layer) is greater than the first mode pore size (the mode pore size of the first layer). In some embodiments, the second mode pore size (the mode pore size of the second layer) is smaller than the first mode pore size (the mode pore size of the first layer).

In some embodiments, the second mode pore size is greater than or equal to 2 microns, greater than or equal to 3 microns, greater than or equal to 4 microns, greater than or equal to 5 microns, greater than or equal to 10 microns, or greater than or equal to 15 microns. In some embodiments, the first mode pore size is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 2 microns and less than or equal to 30 microns or greater than or equal to 10 microns and less than or equal to 20 microns).

In some embodiments, a certain percentage of the pores of the second layer are below a certain size. In some embodiments, the certain percentage is greater than or equal to 20%, greater than or equal to 30%, greater than or equal to 40%, greater than or equal to 50%, greater than or equal to 60%, greater than or equal to 70%, greater than or equal to 80%, or greater than or equal to 90% of the pores of the second layer are below a certain size. In some embodiments, the certain percentage is less than or equal to 100%, less than or equal to 90%, less than or equal to 80%, less than or equal to 70%, less than or equal to 60%, less than or equal to 50%, less than or equal to 40%, or less than or equal to 30% of the pores of the second layer are below a certain size. Combinations of these ranges are also possible (e.g., greater than or equal to 20% and less than or equal to 100%, greater than or equal to 50% and less than or equal to 100%, or greater than or equal to 90% and less than or equal to 100%). In some embodiments, the certain size of the pores is greater than or equal to 2 microns, greater than or equal to 3 microns, greater than or equal to 4 microns, greater than or equal to 5 microns, greater than or equal to 10 microns, or greater than or equal to 15 microns. In some embodiments, the certain size of the pores is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 2 microns and less than or equal to 30 microns or greater than or equal to 10 microns and less than or equal to 20 microns). For example, in some embodiments, greater than or equal to 20% (e.g., greater than or equal to 50% or greater than or equal to 90%) of the pores of the second layer have a pore size of less than or equal to 20 microns (e.g., greater than or equal to 10 microns and less than or equal to 20 microns).

In some embodiments, the second layer comprises a first surface and a second surface. In some embodiments, the first surface faces the first layer (e.g., is directly adjacent to a surface of the first layer). In some embodiments, the second surface faces the third layer (e.g., is directly adjacent to a surface of the third layer). For example, in some embodiments, second layer 120 in FIG. 4 comprises first surface 121, which faces first layer 110, and second surface 122, which faces third layer 130.

In some embodiments, the first surface has a mode pore size. In some embodiments, the mode pore size of the first surface is greater than or equal to 10 microns, greater than or equal to 15 microns, or greater than or equal to 20 microns. In some embodiments, the mode pore size of the first surface is less than or equal to 35 microns, less than or equal to 30 microns, or less than or equal to 25 microns. Combinations of these ranges are also possible (e.g., greater than or equal to 10 microns and less than or equal to 35 microns, greater than or equal to 15 microns and less than or equal to 25 microns, or greater than or equal to 20 microns and less than or equal to 25 microns).

In some embodiments, the second surface has a mode pore size. In some embodiments, the mode pore size of the second surface is greater than or equal to 0.01 microns, greater than or equal to 0.05 microns, greater than or equal to 0.1 microns, greater than or equal to 0.15 microns, greater than or equal to 0.25 microns, greater than or equal to 0.5 microns, or greater than or equal to 1 micron. In some embodiments, the mode pore size of the second surface is less than or equal to 5 microns, less than or equal to 3 microns, less than or equal to 1 micron, less than or equal to 0.5 microns, less than or equal to 0.3 microns, or less than or equal to 0.2 microns. Combinations of these ranges are also possible (e.g., greater than or equal to 0.01 microns and less than or equal to 1 micron, greater than or equal to 0.1 microns and less than or equal to 0.2 microns, or greater than or equal to 0.1 microns and less than or equal to 5 microns).

In some embodiments, the mode pore size of the second surface (e.g., the surface facing the third layer) is smaller than the mode pore size of the first surface (e.g., the surface facing the first layer). In some embodiments, the ratio of the mode pore size of the first surface to the mode pore size of the second surface is greater than or equal to 5:1, greater than or equal to 10:1, greater than or equal to 25:1, greater than or equal to 50:1, greater than or equal to 75:1, greater than or equal to 100:1, greater than or equal to 125:1, or greater than or equal to 150:1. In some embodiments, the ratio of the mode pore size of the first surface to the mode pore size of the second surface is less than or equal to 1,000:1, less than or equal to 500:1, less than or equal to 250:1, less than or equal to 200:1, less than or equal to 175:1, less than or equal to 150:1, less than or equal to 125:1, less than or equal to 100:1, less than or equal to 75:1, or less than or equal to 50:1. Combinations of these ranges are also possible (e.g., greater than or equal to 5:1 and less than or equal to 1,000:1, greater than or equal to 100:1 and less than or equal to 200:1, greater than or equal to 125:1 and less than or equal to 175:1, or greater than or equal to 150:1 and less than or equal to 175:1).

Mode pore size can be measured using any suitable technique. For example, in some embodiments, mode pore size can be measured using Mercury Intrusion Porosimetry or Scanning Electron Microscope (SEM). In some embodiments, mode pore size can be measured over the full thickness of the layer. In some embodiments, a layer can be divided into multiple sections along the thickness of the layer, and the mode pore size of each section can be measured.

In some embodiments, the first surface and/or the second surface each independently have a thickness that is a certain percentage of the thickness of the second layer. In some embodiments, the first surface and/or the second surface are each independently greater than or equal to 1/10 of the thickness of the second layer, greater than or equal to ⅛ of the thickness of the second layer, greater than or equal to ⅙ of the thickness of the second layer, or greater than or equal to 1/10 of the thickness of the second layer ¼ of the thickness of the second layer. In some embodiments, the first surface and/or second surface are each independently less than or equal to ½ of the thickness of the second layer, less than or equal to ⅓ of the thickness of the second layer, less than or equal to ¼ of the thickness of the second layer, or less than or equal to ⅕ of the thickness of the second layer. Combinations of these ranges are also possible (e.g., greater than or equal to 1/10 of the thickness of the second layer and less than or equal to ½ of the thickness of the second layer, or greater than or equal to ⅛ of the thickness of the second layer and less than or equal to ¼ of the thickness of the second layer). In some embodiments, the first surface and the second surface have the same thickness.

In some embodiments, the second layer has a gradient in mode pore size between the first surface and the second surface. In some embodiments, there are cross-sections within the thickness of the second layer between the first surface and the second surface. In some embodiments the cross-sections have a mode pore size that is between the mode pore size of the first surface and the mode pore size of the second surface. For example, in that embodiment, if the mode pore size of the first surface was 11 microns and the mode pore size of the second surface was 1 micron, then the cross-sections within the thickness of the second layer between the first surface and the second surface would have mode pore sizes between 1 micron and 11 microns.

In some embodiments, the second layer can have a variety of suitable thicknesses. In some embodiments, the thickness of the second layer is greater than or equal to 100 microns. In some embodiments, the thickness of the second layer is less than or equal to 300 microns, less than or equal to 250 microns, less than or equal to 200 microns, or less than or equal to 150 microns. Combinations of these ranges are also possible (e.g., greater than or equal to 100 microns and less than or equal to 150 microns, or greater than or equal to 100 microns and less than or equal to 300 microns).

In some embodiments, the second layer has a relatively low absorbency. In some embodiments, the absorbency of the second layer is less than or equal to 50 microliters/cm², less than or equal to 40 microliters/cm², less than or equal to 30 microliters/cm², less than or equal to 25 microliters/cm², less than or equal to 20 microliters/cm², less than or equal to 15 microliters/cm², less than or equal to 10 microliters/cm², or less than or equal to 5 microliters/cm². In some embodiments, the absorbency of the second layer is greater than or equal to 10 microliters/cm², greater than or equal to 15 microliters/cm², or greater than or equal to 20 microliters/cm². Combinations of these ranges are also possible (e.g., greater than or equal to 10 microliters/cm² and less than or equal to 50 microliters/cm², or greater than or equal to 15 microliters/cm² and less than or equal to 25 microliters/cm²). In some embodiments, the relatively low absorbency of the second layer increases the separation efficiency and/or the volume of sample recovered (e.g., increases the yield of the separation), as a lower volume of the blood plasma is retained by the second layer.

In some embodiments, the article comprises a third layer (e.g., absorbent layer). In some embodiments, the third layer (e.g., absorbent layer) comprises a wicking source. In some embodiments, the third layer (e.g., absorbent layer) comprises rayon and/or polyester (e.g., Kapmat). In some embodiments, the third layer (e.g., absorbent layer) comprises a blend of rayon and polyester, or a blend of rayon and polypropylene (e.g., ShamWow). The third layer (e.g., absorbent layer) may be fibrous or non-fibrous.

In some embodiments, the third layer (e.g., absorbent layer) is porous. In some embodiments, the third layer (e.g., absorbent layer) has a third mode pore size. In some embodiments, the third mode pore size is greater than or equal to 20 microns, greater than or equal to 30 microns, greater than or equal to 40 microns, greater than or equal to 50 microns, greater than or equal to 60 microns, greater than or equal to 70 microns, greater than or equal to 75 microns, greater than or equal to 80 microns, or greater than or equal to 90 microns. In some embodiments, the third mode pore size is less than or equal to 150 microns, less than or equal to 140 microns, less than or equal to 130 microns, less than or equal to 125 microns, less than or equal to 120 microns, less than or equal to 110 microns, or less than or equal to 100 microns. Combinations of these ranges are also possible (e.g., greater than or equal to 20 microns and less than or equal to 150 microns, greater than or equal to 75 microns and less than or equal to 125 microns, or greater than or equal to 90 microns and less than or equal to 100 microns).

In some embodiments, the third layer (e.g., absorbent layer) may have a relatively large absorbency. In some embodiments, the absorbency is greater than or equal to 55 microliters/cm², greater than or equal to 60 microliters/cm², greater than or equal to 65 microliters/cm², greater than or equal to 70 microliters/cm², greater than or equal to 75 microliters/cm², greater than or equal to 80 microliters/cm², greater than or equal to 85 microliters/cm², greater than or equal to 90 microliters/cm², greater than or equal to 95 microliters/cm², greater than or equal to 100 microliters/cm², greater than or equal to 125 microliters/cm², greater than or equal to 150 microliters/cm², greater than or equal to 175 microliters/cm², greater than or equal to 200 microliters/cm², greater than or equal to 250 microliters/cm², greater than or equal to 300 microliters/cm², or greater than or equal to 400 microliters/cm². In some embodiments, the absorbency is less than or equal to 600 microliters/cm², less than or equal to 550 microliters/cm², less than or equal to 500 microliters/cm², less than or equal to 450 microliters/cm², less than or equal to 400 microliters/cm², less than or equal to 300 microliters/cm², less than or equal to 250 microliters/cm², less than or equal to 200 microliters/cm², less than or equal to 175 microliters/cm², or less than or equal to 150 microliters/cm². Combinations of these ranges are also possible (e.g., greater than or equal to 80 microliters/cm² and less than or equal to 600 microliters/cm², greater than or equal to 100 microliters/cm² and less than or equal to 600 microliters/cm², or greater than or equal to 200 microliters/cm² and less than or equal to 450 microliters/cm²).

As used herein, the absorbency of an article and/or layer is determined by weighing the article and/or layer, saturating it in DI water for 30 seconds at room temperature, weighing it again, determining the difference between the second weight and the first weight (i.e., the weight of the DI water absorbed), and then converting this weight to a volume of water (e.g., microliters) using the density of DI water at room temperature. The volume of DI water absorbed is then normalized by dividing by the surface area (e.g., cm²) of the article and/or layer.

In some embodiments, the relatively large absorbency of the third layer (e.g., absorbent layer) facilitates passive separation by increasing capillary action and/or facilitates collection and/or storage of the absorbed fluid in the third layer (e.g., absorbent layer).

In some embodiments, the third layer (e.g., absorbent layer) is configured to absorb a variety of suitable fluids. Examples of suitable fluids include water, blood plasma, saliva, urine, wound exudate, and/or cerebrospinal fluid. In some embodiments, the third layer (e.g., absorbent layer) is configured to absorb blood plasma.

In some embodiments, the third layer (e.g., absorbent layer) may have a relatively large release. As used herein, the release of an article and/or layer is the percentage of the absorbed water (determined as described above) that is released upon centrifugation. Once the article and/or layer is saturated in DI water for 30 seconds and the volume of DI water absorbed is calculated (as discussed above), the article and/or layer is centrifuged at an RCF of 800 g for 5 minutes. The volume of DI water released during centrifugation is then converted to a percentage of the volume of DI water that was absorbed in order to determine what percentage of the absorbed DI water was released. This value is the release of the article and/or layer.

In some embodiments, the third layer (e.g., absorbent layer) has a release that is greater than or equal to 15%, greater than or equal to 20%, greater than or equal to 25%, greater than or equal to 30%, greater than or equal to 35%, greater than or equal to 40%, greater than or equal to 50%, greater than or equal to 60%, greater than or equal to 70%, greater than or equal to 80%, or greater than or equal to 90%. In some embodiments, the third layer has a release that is less than or equal to 100%, less than or equal to 95%, less than or equal to 90%, less than or equal to 85%, less than or equal to 80%, less than or equal to 75%, less than or equal to 70%, or less than or equal to 60%. Combinations of these ranges are also possible (e.g., greater than or equal to 35% and less than or equal to 100%, greater than or equal to 50% and less than or equal to 100%, greater than or equal to 70% and less than or equal to 100%, or greater than or equal to 70% and less than or equal to 90%).

In some embodiments, the relatively large release of the third layer (e.g., absorbent layer) increases separation efficiency and/or the volume of sample recovered (e.g., increases the yield of the separation).

In some embodiments, the third layer (e.g., absorbent layer) has a relatively large thickness (e.g., compared to the first and/or second layer(s)). In some embodiments, the thickness of the third layer (e.g., absorbent layer) is greater than or equal to 200 microns, greater than or equal to 225 microns, or greater than or equal to 250 microns. In some embodiments, the thickness of the third layer is less than or equal to 800 microns, less than or equal to 700 microns, less than or equal to 600 microns, or less than or equal to 500 microns. Combinations of these ranges are also possible (e.g., greater than or equal to 200 microns and less than or equal to 800 microns, or greater than or equal to 250 microns and less than or equal to 500 microns). In some embodiments, the relatively large thickness of the third layer increases the volume of sample recovered (e.g., increases the yield of the separation), as it increases the volume of fluid that can be absorbed.

In some embodiments, the article comprises a support structure. For example, in some embodiments, the article in FIG. 5 comprises support structure 204. In some embodiments, the support structure comprises a plastic, an acrylic, and/or a metal. In some embodiments, the support structure is a plastic scaffold or an acrylic scaffold. In some embodiments, the support structure is configured to maintain conformal contact between the third layer and one or more layers (e.g., the second layer).

In some embodiments, the support structure is adjacent one or more layers. In some embodiments, the support structure is adjacent the first layer, second layer, and/or third layer. In some embodiments, the support structure is in direct contact with one or more layers. In some embodiments, the support structure is in direct contact with the first layer, second layer, and/or third layer. In some embodiments, the support structure is in direct contact with the second layer and third layer. In some embodiments, the support structure is in direct contact with the third layer.

In some embodiments, the support structure is adhered to one or more layers (e.g., the third layer (e.g., absorbent layer)). Examples of suitable means to adhere (e.g., the support structure to one or more layers) are discussed elsewhere herein (e.g., in reference to adhering one layer to another layer). In some embodiments, the support structure is not adhered to one or more layers (e.g., not adhered to any layers). For example, in some embodiments, a portion of the article (e.g., the first layer, the second layer, and/or the third layer) sits on the support structure.

In some embodiments, the support structure comprises a cavity. In some embodiments, the cavity is used for holding a portion of the article (e.g., the first layer, the second layer, and/or the third layer). In some embodiments, the cavity is circular, oval, square, rectangular, and/or diamond shaped. In some embodiments, the cavity is of a similar shape as a cross-section (e.g., a horizontal cross-section) of a portion of the article (e.g., one or more layers, such as the third layer). For example, in some embodiments, the cavity and/or the cross-section of a portion of the article (e.g., one or more layers, such as the third layer) are both circular, oval, square, rectangular, and/or diamond shaped.

The first layer, second layer, third layer, and/or article may have any suitable maximum horizontal dimension. In some embodiments, the first layer, second layer, third layer, and/or article each independently have a maximum horizontal dimension of greater than or equal to 4 millimeters, greater than or equal to 6 millimeters, greater than or equal to 7 millimeters, greater than or equal to 8 millimeters, greater than or equal to 10 millimeters, greater than or equal to 12 millimeters, greater than or equal to 14 millimeters, greater than or equal to 16 millimeters, greater than or equal to 18 millimeters, greater than or equal to 20 millimeters, greater than or equal to 40 millimeters, greater than or equal to 60 millimeters, greater than or equal to 80 millimeters, greater than or equal to 100 millimeters, greater than or equal to 120 millimeters, greater than or equal to 140 millimeters, or greater than or equal to 150 millimeters. In some embodiments, the first layer, second layer, third layer, and/or article each independently have a maximum horizontal dimension of less than or equal to 500 millimeters, less than or equal to 400 millimeters, less than or equal to 300 millimeters, less than or equal to 200 millimeters, less than or equal to 180 millimeters, less than or equal to 160 millimeters, less than or equal to 140 millimeters, less than or equal to 120 millimeters, less than or equal to 100 millimeters, less than or equal to 80 millimeters, less than or equal to 60 millimeters, less than or equal to 40 millimeters, less than or equal to 20 millimeters, less than or equal to 18 millimeters, less than or equal to 16 millimeters, less than or equal to 14 millimeters, less than or equal to 12 millimeters, less than or equal to 10 millimeters, or less than or equal to 9 millimeters. Combinations of these ranges are also possible (e.g., greater than or equal to 4 millimeters and less than or equal to 500 millimeters, greater than or equal to 4 millimeters and less than or equal to 16 millimeters, greater than or equal to 6 millimeters and less than or equal to 20 millimeters, greater than or equal to 20 millimeters and less than or equal to 500 millimeters, greater than or equal to 20 millimeters and less than or equal to 100 millimeters, greater than or equal to 60 millimeters and less than or equal to 200 millimeters, greater than or equal to 8 millimeters and less than or equal to 16 millimeters, greater than or equal to 10 millimeters and less than or equal to 20 millimeters, or greater than or equal to 7 millimeters and less than or equal to 9 millimeters). In some embodiments, the maximum horizontal dimensions of one or more (e.g., two or three) of the first layer, second layer, and third layer are the same.

In some embodiments, a maximum horizontal dimension of one or more layers (e.g., the second layer, or all of the layers) larger than a lower limit disclosed herein reduces clogging, reduces hemolysis, increases separation efficiency, decreases the separation time, increases the volume of sample recovered (e.g., increases the yield of the separation), and/or increases input volume. For example, having a maximum horizontal dimension of the second layer larger than a lower limit disclosed herein reduces clogging of the pores (increasing separation efficiency, decreasing separation time, increasing the volume of sample recovered, increasing the yield of the separation, and/or increasing input volume) and reduces hemolysis, in some cases.

In some instances, a maximum horizontal dimension of one or more layers (e.g., the absorbent layer, or all of the layers) smaller than an upper limit disclosed herein increases the volume of sample recovered (e.g., increases the yield of separation). For example, for the absorbent layer, in some cases, having a maximum horizontal dimension lower than an upper limit disclosed herein provides greater saturation of the absorbent material by a given sample (e.g., blood plasma), which, in some instances, provides increased volume of sample recovered and increased yield of separation (e.g., when compressed, more of the sample is released rather than being redistributed within the absorbent layer to unsaturated portions). As another example, for the first layer, having a maximum horizontal dimension smaller than an upper limit disclosed herein reduces the void volume, which, for a given sample input volume, allows increased volume of sample recovered and increased yield, in some instances.

In some embodiments, the maximum horizontal dimension of the cavity is greater than or equal to the maximum horizontal dimension of a portion of the article (e.g., one or more layers, such as the second layer and/or the third layer). In some embodiments, the ratio of the maximum horizontal dimension of the cavity to the maximum horizontal dimension of a portion of the article (e.g., one or more layers, such as the second layer and/or the third layer) is greater than or equal to 1:1, greater than or equal to 1.05:1, greater than or equal to 1.1:1, greater than or equal to 1.2:1, greater than or equal to 1.3:1, greater than or equal to 1.4:1, or greater than or equal to 1.5:1. In some embodiments, the ratio of the maximum horizontal dimension of the cavity to the maximum horizontal dimension of a portion of the article (e.g., one or more layers, such as the second layer and/or the third layer) is less than or equal to 3:1, less than or equal to 2:1, less than or equal to 1.5:1, less than or equal to 1.4:1, less than or equal to 1.3:1, less than or equal to 1.2:1, less than or equal to 1.1:1, or less than or equal to 1.05:1. Combinations of these ranges are also possible (e.g., greater than or equal to 1:1 and less than or equal to 3:1 or greater than or equal to 1.1 and less than or equal to 1.3:1).

In some embodiments, the maximum horizontal dimension of the cavity is greater than or equal to 0.5 cm, greater than or equal to 0.75 cm, greater than or equal to 1 cm, greater than or equal to 1.1 cm, greater than or equal to 1.2 cm, greater than or equal to 1.3 cm, greater than or equal to 1.4 cm, greater than or equal to 1.5 cm, greater than or equal to 1.6 cm, greater than or equal to 1.7 cm, greater than or equal to 1.8 cm, greater than or equal to 1.9 cm, greater than or equal to 2 cm, greater than or equal to 2.25 cm, greater than or equal to 2.5 cm, or greater than or equal to 3 cm. In some embodiments, the maximum horizontal dimension of the cavity is less than or equal to 10 cm, less than or equal to 5 cm, less than or equal to 4 cm, less than or equal to 3 cm, less than or equal to 2.5 cm, less than or equal to 2.25 cm, less than or equal to 2 cm, less than or equal to 1.9 cm, less than or equal to 1.8 cm, less than or equal to 1.7 cm, less than or equal to 1.6 cm, less than or equal to 1.5 cm, less than or equal to 1.4 cm, less than or equal to 1.3 cm, less than or equal to 1.2 cm, less than or equal to 1.1 cm, or less than or equal to 1 cm. Combinations of these ranges are also possible (e.g., greater than or equal to 0.5 cm and less than or equal to 10 cm or greater than or equal to 0.5 cm and less than or equal to 2 cm).

In some embodiments, the depth of the cavity is less than the thickness of the support structure, such that, when viewed from above, a layer of the support structure is present throughout the surface area of the support structure. In some embodiments, the cavity is configured such that a portion of the article (e.g., the first layer, second layer, and/or third layer) can sit inside the cavity. In some embodiments, the cavity is configured such that a portion of the article (e.g., the first layer, second layer, and/or third layer) can sit inside the cavity, with the bottom surface of the third layer in contact with the support structure.

In some embodiments, the cavity is present throughout the thickness of the support structure, such that, when viewed from above, the cavity is a hole in the support structure. In some embodiments, the cavity has different maximum horizontal dimensions at different thickness of the support structure. For example, in some embodiments, the cavity has a larger maximum horizontal dimension at one opening than at the other. In some embodiments, the larger maximum horizontal dimension at one opening is greater than or equal to the maximum horizontal dimension of a portion of the article (e.g., the third layer). In some embodiments, the smaller maximum horizontal dimension at the other opening is less than the maximum horizontal dimension of a portion of the article (e.g., the third layer). In some embodiments, the cavity is configured such that a portion of the article (e.g., the first layer, second layer, and/or third layer) can sit inside the cavity. In some embodiments, the cavity is configured such that a portion of the article (e.g., the first layer, second layer, and/or third layer) can sit inside the cavity, but the bottom surface of the third layer is not in contact with the support structure. In some embodiments, the cavity is configured such that a portion of the article (e.g., the first layer, second layer, and/or third layer) can sit inside the cavity, but the bottom surface of the third layer is not in contact with the support structure, such that the third layer can be removed from the article through the bottom of the support structure (e.g., through the opening with the smaller maximum horizontal dimension), while the remaining portions of the article can remain in the support structure (see, e.g., FIG. 6).

In some embodiments, the cavity is configured such that the height of the edges (e.g., circumference) of the cavity prevent a portion of the article (e.g., the first layer, second layer, and/or third layer) from significant horizontal movement, but the portion of the article (e.g., the first layer, second layer, and/or third layer) can still be picked up vertically. In some embodiments, the height of the edges of the cavity are greater than or equal to ⅕ the thickness of a layer (e.g., the third layer), greater than or equal to ¼ the thickness of a layer (e.g., the third layer), greater than or equal to ⅓ the thickness of a layer (e.g., the third layer), greater than or equal to ½ the thickness of a layer (e.g., the third layer), or greater than or equal to the thickness of a layer (e.g., the third layer). In some embodiments, the height of the edges of the cavity are less than or equal to 3 times the thickness of a layer (e.g., the third layer), 2 times the thickness of a layer (e.g., the third layer), the thickness of a layer (e.g., the third layer), ½ the thickness of a layer (e.g., the third layer), ⅓ the thickness of a layer (e.g., the third layer), or ¼ the thickness of a layer (e.g., the third layer). Combinations of these ranges are also possible (e.g., greater than or equal to ⅕ and less than or equal to 3 times the thickness of a layer (e.g., the third layer)).

The layers in the article may be in any suitable order. In some embodiments, the first layer is positioned between the second layer and third layer. In some embodiments, the third layer is positioned between the first layer and second layer. In some embodiments, the second layer is positioned between the first layer and the third layer (e.g., absorbent layer). For example, in FIG. 4, in accordance with some embodiments, second layer 120 is positioned between first layer 110 and third layer 130.

In some embodiments, there are no intervening layers between the first layer and second layer and/or between the second layer and third layer (e.g., absorbent layer). For example, in FIG. 4, in accordance with some embodiments, there are no intervening layers between first layer 110 and second layer 120 or between second layer 120 and third layer 130. In some embodiments, the direct contact (e.g., direct conformal contact) between the layers (e.g., between the first layer and second layer and/or between the second layer and third layer) decreases the separation time by increasing capillary action.

In some embodiments, one or more layers are adhered to one or more layers (e.g., the first layer is adhered to the second layer). For example, in some embodiments, the article in FIG. 5 comprises adhesive 201, which adheres first layer 200 to second layer 202, and adhesive 203, which adheres second layer 202 to third layer 205. In some embodiments, one or more layers are permanently adhered or integrally connected to one or more layers. In some embodiments, one or more layers are reversibly adhered to one or more layers. Examples of suitable methods of adhering layers include double-sided adhesive (e.g., double-sided medical adhesive), liquid adhesive, sonic welding, and/or compression. In some embodiments, one or more layers are adhered to one or more layers (and/or a support structure) with an adhesive. Examples of suitable adhesives include double-sided adhesive (e.g., double-side medical adhesive), compression tape, 3M brand adhesive, and/or Flexcon brand adhesive. In some embodiments, the adhesive is placed on a surface of a layer. In some embodiments, the adhesive is placed around the perimeter of a layer (e.g., first layer) where it contacts another layer (or substrate) (e.g., second layer) to adhere it to the other layer (or substrate). In some embodiments, the adhesive (e.g., between two layers, or between a layer and the substrate) provides a full seal (e.g., a seal around the entire perimeter of the layer through which fluid cannot pass).

In some embodiments, a full seal (e.g., with adhesive) between one or more layers (and/or between a layer and the substrate) increases the purity of the purified blood (e.g., purified plasma), as it reduces or prevent one or more impurities (e.g., red blood cells) from bypassing one or more layers and entering the third layer. For example, if there was a partial seal around the perimeter of the first layer where it contacts the second layer, then a blood sample might pass through the first layer and out through the holes in the seal, such that it then passes down to the third layer without passing through the second layer, resulting in higher levels of impurities (e.g., red blood cells) than if the blood sample had passed through the second layer.

In some embodiments, the adhesive has any suitable thickness. In some embodiments, the adhesive is relatively thin. In some embodiments, a thin adhesive allows the layers to be closer together, decreasing the separation time. In some embodiments, the adhesive has a thickness of greater than or equal to 0.03 millimeters, greater than or equal to 0.04 millimeters, greater than or equal to 0.05 millimeters, greater than or equal to 0.06 millimeters, or greater than or equal to 0.063 millimeters. In some embodiments, the adhesive has a thickness of less than or equal to 0.2 millimeters, less than or equal to less than or equal to 0.18 millimeters, less than or equal to 0.16 millimeters, less than or equal to 0.14 millimeters, or less than or equal to 0.126 millimeters. Combinations of these ranges are also possible (e.g., greater than or equal to 0.03 millimeters and less than or equal to 0.2 millimeters, or greater than or equal to 0.063 millimeters and less than or equal to 0.126 millimeters).

In some embodiments, the adhesive is applied manually. In some embodiments, the adhesive is applied with a laser cutter, ultrasonic welding, and/or UV curing. In some embodiments, the adhesive has a low tack. In some embodiments, one or more layers is adhered to one or more layers in such a way that they cannot be pulled apart manually without damaging one or more of the layers. For example, in some embodiments, the first layer is adhered to the second layer such that they cannot be pulled apart manually without damaging one or more of the layers. In some embodiments, one or more layers is adhered to one or more layers in such a way that they can be pulled apart manually without damaging one or more of the layers. For example, in some embodiments, the second layer is adhered to the third layer in such a way that they can be pulled apart manually without damaging one or more of the layers (e.g., the third layer). In some embodiments, the second layer is adhered to the third layer in such a way that they can be pulled apart manually, without having to use so much force that it will disrupt the first layer (e.g., creating mess or contamination), but such that the second layer and third layer do not come apart during use (e.g., do not come apart during separation of a blood sample).

In some embodiments, the layers are stacked coaxially, such that a vertical stack is formed. For example, in some embodiments, article 100 in FIG. 4 comprises first layer 110, second layer 120, and third layer 130 stacked coaxially, such that a vertical stack is formed. In some embodiments, the vertical stacking reduces the time required for separation.

In some embodiments, the layers described herein are discrete layers. In some embodiments, the layers described herein are not discrete layers, such that a layer is instead one of multiple phases within a discrete layer. For example, in some embodiments, the first layer and the second layer could be two phases within one layer.

In some embodiments, the maximum horizontal dimension of the article is greater than or equal to 0.5 cm, greater than or equal to 0.75 cm, greater than or equal to 1 cm, greater than or equal to 1.1 cm, greater than or equal to 1.2 cm, greater than or equal to 1.3 cm, greater than or equal to 1.4 cm, greater than or equal to 1.5 cm, greater than or equal to 1.6 cm, greater than or equal to 1.7 cm, greater than or equal to 1.8 cm, greater than or equal to 1.9 cm, greater than or equal to 2 cm, greater than or equal to 2.25 cm, greater than or equal to 2.5 cm, or greater than or equal to 3 cm. In some embodiments, the maximum horizontal dimension of the article is less than or equal to 10 cm, less than or equal to 5 cm, less than or equal to 4 cm, less than or equal to 3 cm, less than or equal to 2.5 cm, less than or equal to 2.25 cm, less than or equal to 2 cm, less than or equal to 1.9 cm, less than or equal to 1.8 cm, less than or equal to 1.7 cm, less than or equal to 1.6 cm, less than or equal to 1.5 cm, less than or equal to 1.4 cm, less than or equal to 1.3 cm, less than or equal to 1.2 cm, less than or equal to 1.1 cm, or less than or equal to 1 cm. Combinations of these ranges are also possible (e.g., greater than or equal to 0.5 cm and less than or equal to 5 cm or greater than or equal to 0.5 cm and less than or equal to 2 cm).

In some embodiments, the article has a high loading capacity (e.g., for whole blood). As used herein, loading capacity is defined as volume of fluid that can be loaded divided by the surface area of the article. In some embodiments, the loading capacity of the article is greater than or equal to 20 microliters/cm², greater than or equal to 30 microliters/cm², greater than or equal to 40 microliters/cm², greater than or equal to 50 microliters/cm², greater than or equal to 60 microliters/cm², greater than or equal to 70 microliters/cm², greater than or equal to 80 microliters/cm², greater than or equal to 90 microliters/cm², greater than or equal to 100 microliters/cm², or greater than or equal to 125 microliters/cm². In some embodiments, the loading capacity of the article is less than or equal to 500 microliters/cm², less than or equal to 400 microliters/cm², less than or equal to 300 microliters/cm², less than or equal to 250 microliters/cm², less than or equal to 200 microliters/cm², less than or equal to 150 microliters/cm², less than or equal to 125 microliters/cm², less than or equal 100 microliters, less than or equal 90 microliters/cm², less than or equal 80 microliters/cm², or less than or equal 70 microliters/cm². Combinations of these ranges are also possible (e.g., greater than or equal to 20 microliters/cm² and less than or equal to 500 microliters/cm², or greater than or equal to 50 microliters/cm² and less than or equal to 150 microliters/cm²).

Methods are described herein. In accordance with some embodiments, an illustrative method is illustrated schematically in FIG. 6, and can be understood in view of FIG. 4.

In some embodiments, the method comprises passing a blood sample across a first layer. For example, in some embodiments, the method comprises passing a blood sample across first layer 110 in FIG. 4. In some embodiments, the first layer comprises any embodiment of the first layer, or combinations thereof, disclosed herein.

In some embodiments, the blood sample is whole blood. In some embodiments, the blood sample is diluted with water and/or a buffer solution. In some embodiments, the blood sample is undiluted blood (e.g., undiluted whole blood) from a subject. In some embodiments, the subject is an animal, such as a mammal. In some embodiments, the subject is a human. In some embodiments, the article comprises an anti-coagulant (e.g., ethylenediaminetetraacetic acid (EDTA) and/or heparin), such as a dried anti-coagulant.

In some embodiments, the first layer has a high loading capacity, such that the blood sample passed across the first layer (e.g., input volume) has a substantial volume. In some embodiments, the volume of the blood sample passed across the first layer (e.g., input volume) is greater than or equal to 25 microliters, greater than or equal to 30 microliters, greater than or equal to 40 microliters, greater than or equal to 50 microliters, greater than or equal to 60 microliters, greater than or equal to 70 microliters, greater than or equal to 80 microliters, greater than or equal to 90 microliters, greater than or equal to 100 microliters, greater than or equal to 125 microliters, greater than or equal to 150 microliters, greater than or equal to 200 microliters, or greater than or equal to 250 microliters. In some embodiments, the volume of the blood sample passed across the first layer (e.g., input volume) is less than or equal to 500 microliters, less than or equal to 400 microliters, less than or equal to 300 microliters, less than or equal to 250 microliters, less than or equal to 200 microliters, less than or equal to 150 microliters, less than or equal to 125 microliters, less than or equal 100 microliters, less than or equal 90 microliters, less than or equal 80 microliters, or less than or equal 70 microliters. Combinations of these ranges are also possible (e.g., greater than or equal to 25 microliters and less than or equal to 500 microliters, greater than or equal to 50 microliters and less than or equal to 300 microliters, or greater than or equal to 100 microliters and less than or equal to 250 microliters).

In some embodiments, the volume of the blood sample passed across the first layer (e.g., input volume) may affect the volume of sample (e.g., plasma) recovered, the separation efficiency, the separation time, and/or the purity (e.g., levels of hemolysis) of the sample (e.g., plasma). For example, if the volume of the blood sample passed across the first layer (e.g., input volume) is too low, then a larger percentage of the blood sample may be absorbed by the first layer and/or second layer resulting in low volume of sample recovered (e.g., low yield of the separation) and/or low separation efficiency compared to if a larger volume of the blood sample passed across the first layer (e.g., input volume), in some embodiments. As another example, if the volume of the blood sample passed across the first layer (e.g., input volume) is too high, then one or more layers may clog, resulting in more impurities passing through, increased hemolysis, and/or decreased separation time, in some embodiments.

In some embodiments, passing the blood sample across the first layer produces a blood sample with reduced red blood cells. In some embodiments, the red blood cells are reduced by the first layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the red blood cells are reduced by the first layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 90%).

In some embodiments, the first layer reduces the level of red blood cells in the blood sample by size exclusion and/or electrostatic interactions.

In some embodiments, the first layer reduces the level of white blood cells (which can also be called “leukocytes”). In some embodiments, the white blood cells are reduced by the first layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the white blood cells are reduced by the first layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 90%).

In some embodiments, the first layer reduces the level of white blood cells in the blood sample by size exclusion, electrostatic interactions, and/or adsorption of the white blood cells.

In some embodiments, use of the first layer facilitates quick removal of a significant portion of the red blood cells (and/or white blood cells), such that the second layer is less likely to get clogged and/or is less likely to cause hemolysis and/or the article can have a higher loading capacity without requiring lengthy times for separation.

In some embodiments, the method comprises passing the blood sample with reduced red blood cells (and/or white blood cells) across a second layer. For example, in some embodiments, the method comprises passing the blood sample with reduced red blood cells (and/or white blood cells) across second layer 120 in FIG. 4. In some embodiments, the second layer comprises any embodiment of the second layer, or combinations thereof, disclosed herein.

In some embodiments, passing the blood sample with reduced red blood cells (and/or white blood cells) across the second layer produces a blood sample with further reduced red blood cells. In some embodiments, the red blood cells are reduced by the second layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample with reduced red blood cells. In some embodiments, the red blood cells are reduced by the second layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 90%).

In some embodiments, the second layer further reduces the level of red blood cells in the blood sample with reduced red blood cells (and/or white blood cells) by size exclusion and/or electrostatic interactions.

In some embodiments, the second layer reduces the level of white blood cells. In some embodiments, the white blood cells are reduced by the second layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample with reduced red blood cells. In some embodiments, the white blood cells are reduced by the second layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 90%).

In some embodiments, the second layer reduces the level of white blood cells in the blood sample with reduced red blood cells by size exclusion and/or electrostatic interactions.

In some embodiments, use of a second layer with a gradient in pore size reduces the risk of the second layer clogging and/or reduces the risk that the second layer will result in hemolysis, in some embodiments.

In some embodiments, the method comprises passing the blood sample with further reduced red blood cells into a third layer. For example, in some embodiments, the method comprises passing a blood sample with further reduced red blood cells into third layer 130 in FIG. 4. In some embodiments, the third layer comprises any embodiment of the third layer, or combinations thereof, disclosed herein.

In some embodiments, the method (e.g., passing the blood sample across the first layer, passing the blood sample with reduced red blood cells across the second layer, passing the blood sample across the separation device, passing the blood sample with further reduced red blood cells into the third layer (e.g., absorbent layer) and/or collecting the blood sample with reduced number of red blood cells in the vessel) is passive. For example, in some embodiments, the method is done solely with the use of gravity and/or capillary action. In some embodiments, the method is done without the use of centrifugation, electricity, vacuum, and/or an external field (e.g., acoustic, electric, and/or magnetic). For example, in some embodiments, FIG. 6 demonstrates adding blood sample to the article (e.g., the first layer) and then the article separates the sample without further action (that is, the sample is separated purely from gravity and capillary action).

In some embodiments, a portion of the method (e.g., passing the blood sample across the separation device, passing the blood sample across the first layer, passing the blood sample with reduced red blood cells across the second layer, and/or passing the blood sample with further reduced red blood cells into the third layer (e.g., absorbent layer)) is relatively rapid as the separation time is short. In some embodiments, a portion of the method is accomplished within (and/or the separation time is) less than or equal to 30 minutes, less than or equal to 20 minutes, less than or equal to 15 minutes, less than or equal to 10 minutes, less than or equal to 5 minutes, less than or equal to 3 minutes, or less than or equal to 2 minutes. In some embodiments, a portion of the method is accomplished within (and/or the separation time is) greater than or equal to 30 seconds, greater than or equal to 1 minute, greater than or equal to 2 minutes, greater than or equal to 3 minutes, or greater than or equal to 5 minutes. Combinations of these ranges are also possible (e.g., greater than or equal to 30 seconds and less than or equal to 10 minutes or greater than or equal to 30 seconds and less than or equal to 5 minutes).

In some embodiments, the method (e.g., passing the blood sample across the separation device, passing the blood sample across the first layer, passing the blood sample with reduced red blood cells across the second layer, passing the blood sample with further reduced red blood cells into the third layer, and/or collecting the blood sample with reduced number of red blood cells in the vessel) has a high separation efficiency. In some embodiments, the separation efficiency is greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, or greater than or equal to 55%. In some embodiments, the separation efficiency is less than or equal to 100%, less than or equal to 90%, less than or equal to 80%, less than or equal to 70%, less than or equal to 60%, less than or equal to 55%, less than or equal to 50%, less than or equal to 45%, less than or equal to 40%, less than or equal to 35%, or less than or equal to 30%. Combinations of these ranges are also possible (e.g., greater than or equal to 10% and less than or equal to 100%, greater than or equal to 10% and less than or equal to 60%, or greater than or equal to 30% and less than or equal to 55%).

As used herein, the separation efficiency is the percentage of collected purified plasma volume (or volume of purified plasma that passes into the third layer) compared to the total theoretical plasma volume. The total theoretical plasma volume is based on the measured hematocrit value and input sample volume. For example, if a 100 microliter sample has a measured hematocrit value of 50%, then the total theoretical plasma volume is 50 microliters. If 40 microliters of purified plasma were collected (or passed into the third layer), the separation efficiency would be 80%, since 40 microliters is 80% of 50 microliters.

In some embodiments, the method comprises removing the third layer from the second layer. For example, in some embodiments, FIG. 6 demonstrates removing the third layer from the second layer. In some embodiments, the third layer is removed from the second layer by pulling it apart from the second layer. In some embodiments, the third layer is pulled apart from the second layer manually (e.g., pulling it apart with tweezers). In some embodiments, the article comprises a tab. In some embodiments, pulling the tab may pull the third layer apart from the second layer.

In some embodiments, the blood sample with further reduced red blood cells is used directly from the third layer (e.g., absorbent layer). For example, in some embodiments, the third layer can be used as a stamp with which to apply the blood sample with further reduced red blood cells (e.g., to a lateral flow test).

In some embodiments, the blood sample with further reduced red blood cells is stored inside the third layer (e.g., absorbent layer). In some embodiments, the blood sample with further reduced red blood cells is stored inside the third layer in a wet state. In some embodiments, the blood sample with further reduced red blood cells is stored inside the third layer in a dry state. For example, in some embodiments, the third layer containing the blood sample with further reduced red blood cells is dried overnight. In some embodiments, the third layer is dried overnight in a sealed container. In some embodiments, the sealed container comprises a desiccant.

In some embodiments, the dried third layer is later rehydrated. In some embodiments, the dried third layer is rehydrated by adding a solvent, such as an aqueous solution (e.g., an aqueous solution comprising a surfactant), a buffered solution (e.g., phosphate buffered saline), and/or water (e.g., DI water).

In some embodiments, the method comprises collecting the blood sample with further reduced red blood cells from the third layer (e.g., absorbent layer). In some embodiments, collecting the blood sample with further reduced red blood cells is done shortly after the blood sample with further reduced red blood cells is passed into the third layer. In some embodiments, collecting the blood sample with further reduced red blood cells is done after the sample with further reduced blood cells has been stored (e.g., in a wet state or in a dry state) inside the third layer for a length of time. In some embodiments, the blood sample with further reduced red blood cells is collected from the third layer greater than or equal to 1 minute, greater than or equal to 5 minutes, greater than or equal to 15 minutes, greater than or equal to 30 minutes, greater than or equal to 1 hour, greater than or equal to 5 hours, greater than or equal to 12 hours, greater than or equal to 1 day, greater than or equal to 3 days, greater than or equal to 1 week, greater than or equal to 1 month, greater than or equal to 6 months, or greater than or equal to 1 year after it has been passed into the third layer. In some embodiments, the blood sample with further reduced red blood cells is collected from the third layer less than or equal to 3 years, less than or equal to 2 years, less than or equal to 1 year, less than or equal to 6 months, less than or equal to 1 month, less than or equal to 1 week, less than or equal to 3 days, less than or equal to 1 day, less than or equal to 12 hours, less than or equal to 5 hours, less than or equal to 1 hour, less than or equal to 30 minutes, less than or equal to 15 minutes, or less than or equal to 5 minutes after it has been passed into the third layer. Combinations of these ranges are also possible (e.g., greater than or equal to 1 minute and less than or equal to 3 years).

In some embodiments, collecting the blood sample with further reduced red blood cells from the third layer (e.g., absorbent layer) can be accomplished with relatively low amounts of force. In some embodiments, collecting the blood sample with further reduced red blood cells comprises compression (e.g., squeezing) and/or centrifuging the third layer (e.g., with a benchtop centrifuge). For example, in some embodiments, FIG. 6 demonstrates collecting the blood sample with further reduced red blood cells from the third layer by centrifugation with a benchtop centrifuge. In some embodiments, the blood sample is centrifuged at less than or equal to 800 ×g (e.g., less than or equal to 700 ×g, less than or equal to 500 ×g, or less than or equal to 300 ×g) for less than or equal to 5 minutes (e.g., less than or equal to 4 minutes, less than or equal to 3 minutes, less than or equal to 2 minutes, or less than or equal to 1 minute).

In some embodiments, the blood sample with further reduced red blood cells (e.g., the blood sample with further reduced red blood cells collected from the third layer) can be collected in a short period of time. In some embodiments, the blood sample with further reduced blood cells can be collected in less than or equal to 30 minutes, less than or equal to 20 minutes, less than or equal to 15 minutes, less than or equal to 10 minutes, less than or equal to 5 minutes, less than or equal to 3 minutes, or less than or equal to 1 minute. In some embodiments, the blood sample with further reduced blood cells can be collected in greater than or equal to 30 seconds, greater than or equal to 1 minute, greater than or equal to 2 minutes, greater than or equal to 3 minutes, or greater than or equal to 5 minutes. Combinations of these ranges are also possible (e.g., greater than or equal to 30 seconds and less than or equal to 30 minutes, or greater than or equal to 30 seconds and less than or equal to 10 minutes).

In some embodiments the method comprises using the blood sample with further reduced red blood cells (e.g., pure plasma) in subsequent applications (e.g., after collection, and/or directly, from the third layer), such as in a diagnostic health test, a clinical assay (e.g., clinical chemistry assays), an immunoassay, an immunochromatographic assay for antibodies (e.g., tetanus antibodies), quantification of cytokines, amplification of viral RNA, a rapid dipstick test, an HIV viral load assay, a cholesterol test, a metabolite panel, serology for infectious diseases, therapeutic drug monitoring, an ELISA, ICP-AES, HPLC, and/or mass spectrometry.

In some embodiments, the volume of the blood sample with further reduced red blood cells (e.g., the blood sample with further reduced red blood cells collected and/or used directly from the third layer) is a significant percentage of the volume of the blood sample (e.g., the blood sample passed through the first layer), given that 20-60% of the blood sample (e.g., whole blood) is expected to be red blood cells. In some embodiments, the volume of the blood sample with further reduced red blood cells is greater than or equal to 10%, greater than or equal to 12%, greater than or equal to 15%, greater than or equal to 17%, greater than or equal to 20%, greater than or equal to 25%, greater than or equal to 30%, greater than or equal to 35%, greater than or equal to 40%, greater than or equal to 45%, or greater than or equal to 50% of the volume of the blood sample. In some embodiments, the volume of the blood sample with further reduced red blood cells is less than or equal to 80%, less than or equal to 70%, less than or equal to 60%, less than or equal to 50%, less than or equal to 40%, less than or equal to 30%, less than or equal to 25%, less than or equal to 20%, less than or equal to 17%, or less than or equal to 15% of the volume of the blood sample. Combinations of these ranges are also possible (e.g., greater than or equal to 10% and less than or equal to 80% or greater than or equal to 10% and less than or equal to 40%).

In some embodiments, a large volume of the blood sample with further reduced red blood cells is passed into the third layer and/or a large volume of the blood sample with further reduced red blood cells is collected and/or used directly from the third layer. For example, in some embodiments, the volume of the blood sample with further reduced red blood cells passed into the third layer (e.g., absorbent layer) and/or collected and/or used directly from the third layer (e.g., absorbent layer) is greater than or equal to 20 microliters, greater than or equal to 25 microliters, greater than or equal to 30 microliters, greater than or equal to 35 microliters, greater than or equal to 40 microliters, greater than or equal to 45 microliters, greater than or equal to 50 microliters, greater than or equal to 55 microliters, greater than or equal to 60 microliters, greater than or equal to 65 microliters, or greater than or equal to 70 microliters. In some embodiments, the volume of the blood sample with further reduced red blood cells passed into the third layer (e.g., absorbent layer) and/or collected and/or used directly from the third layer (e.g., absorbent layer) is less than or equal to 150 microliters, less than or equal to 125 microliters, less than or equal to 100 microliters, less than or equal to 90 microliters, less than or equal to 80 microliters, less than or equal to 75 microliters, less than or equal to 70 microliters, or less than or equal to 60 microliters. Combinations of these ranges is also possible (e.g., greater than or equal to 20 microliters and less than or equal to 150 microliters, greater than or equal to 30 microliters and less than or equal to 150 microliters, greater than or equal to 50 microliters and less than or equal to 150 microliters, or greater than or equal to 50 microliters and less than or equal to 100 microliters).

In some embodiments, the blood sample with further reduced red blood cells (e.g., the blood sample with further reduced red blood cells collected and/or used directly from the third layer or the blood sample) is pure (e.g., pure plasma and/or serum), substantially free of red blood cells, and/or substantially free of white blood cells. In some embodiments, the blood sample with further reduced red blood cells has less than or equal to 5%, less than or equal to 4%, less than or equal to 3%, less than or equal to 2%, or less than or equal to 1% of the red blood cells in the blood sample (e.g., the original blood sample, such as a whole blood sample). In some embodiments, the blood sample with further reduced red blood cells has less than or equal to 5%, less than or equal to 4%, less than or equal to 3%, less than or equal to 2%, or less than or equal to 1% of the white blood cells in the blood sample (e.g., the original blood sample, such as a whole blood sample).

In some embodiments, the amount of red blood cells is assumed to be the same as the amount of hemoglobin. For example, if a blood sample (e.g., an original blood sample, such as a whole blood sample) had 12 g/dL hemoglobin, and the blood sample with further reduced red blood cells has 0.12 g/dL hemoglobin, then the blood sample with further reduced red blood cells has less than or equal to 1% of the hemoglobin in the original sample, and it would be assumed that the blood sample with further reduced red blood cells has less than or equal to 1% of the red blood cells in the blood sample (e.g., the original blood sample, such as a whole blood sample).

In some embodiments, the blood sample with further reduced red blood cells (e.g., the blood sample with further reduced red blood cells collected and/or used directly from the third layer) has minimal amounts of hemolysis. In some embodiments, the blood sample with further reduced red blood cells has less than or equal to 15% hemolysis, less than or equal to 10% hemolysis, less than or equal to 8% hemolysis, less than or equal to 7%, less than or equal to 6%, less than or equal to 5% hemolysis, less than or equal to 3% hemolysis, less than or equal to 2% hemolysis, or less than or equal to 1% hemolysis. In some embodiments, the blood sample with further reduced red blood cells has greater than or equal to 0% hemolysis, greater than or equal to 0.1% hemolysis, greater than or equal to 0.5% hemolysis, greater than or equal to 1% hemolysis, greater than or equal to 2% hemolysis, greater than or equal to 3% hemolysis, greater than or equal to 4%, or greater than or equal to 5% hemolysis. Combinations of these ranges are also possible (e.g., greater than or equal to 0% and less than or equal to 15% or greater than or equal to 0.1% and less than or equal to 7%).

As used herein, the percentage hemolysis is the percentage of hemoglobin in the measured sample compared to hemoglobin in a similar whole blood sample. For example, if a blood sample was divided in two, and one part was purified (e.g., separated from red blood cells) while the other part was untreated, the percentage hemolysis in the purified sample would be the percentage of hemoglobin in that sample compared to the percentage hemoglobin in the untreated whole blood sample. The amount of hemoglobin can be measured by any suitable assay. For example, the amount of hemoglobin can be measured by the assay described in the example, where a ratio of whole blood (the control) to Drabkin's reagent containing 0.05% (v/v) Brij 25 was 1:250; a ratio of sample to Drabkin's reagent containing 0.05% (v/v) Brij 25 was 1:10; calibration curves were prepared daily using lyophilized hemoglobin standard rehydrated with diH2O (18 MΩ) and diluted over a range 3-20 g/dL; samples were incubated at 21° C. for 15 minutes and absorbance was measured at 540 nm using a microplate reader (e.g., Varioskan LUX).

In some embodiments, the blood sample with further reduced red blood cells (e.g., the blood sample with further reduced red blood cells collected and/or used directly from the third layer) has similar levels of an analyte of interest as the original blood sample (e.g., whole blood and/or the blood sample passed across the first layer). For example, in some embodiments, the level of an analyte of interest in the blood sample with further reduced red blood cells is greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, or greater than or equal to 99% the level of the analyte of interest in the original blood sample (e.g., whole blood and/or the blood sample passed across the first layer). In some embodiments, the level of an analyte of interest in the blood sample with further reduced red blood cells is less than or equal to 100%, less than or equal to 99%, less than or equal to 98%, less than or equal to 95%, less than or equal to 90%, less than or equal to 85%, less than or equal to 80%, less than or equal to 75%, or less than or equal to 70% the level of the analyte of interest in the original blood sample (e.g., whole blood and/or the blood sample passed across the first layer). Combinations of these ranges are also possible (e.g., greater than or equal to 40% and less than or equal to 100% or greater than or equal to 80% and less than or equal to 100%). For example, if a 250 microliter sample of whole blood tested for the presence of HIV RNA by RT-qPCR had an average threshold cycle value of 28 Ct and was passed across an article described herein (e.g., passed across a first layer, passed across a second layer, and passed into a third layer) to form 60 microliters of a blood sample with further reduced red blood cells (e.g., as in a method described herein) with an average threshold cycle value of 29 Ct, then the level of HIV RNA in the blood sample with further reduced red blood cells would be 50% of that in the original blood sample, as every 1 Ct in qPCR is responsible for a doubling.

Examples of analytes of interest may include proteins (e.g., enzymes (e.g., alkaline phosphatase, alanine aminotransferase, aspartate aminotransferase), antibodies (e.g., for immune response (e.g., acute IgM or persistent IgG), such as to indicate vaccination (e.g., measles), infection (e.g., HIV, SARS-CoV-2, tuberculosis, sexually transmitted infections), sensitivity to foods, allergens), and/or biomarkers (e.g., HbA1c, albumin, insulin, cancer antigens (PSA, CA-125))), nucleic acids (e.g., recovered from pathogens (e.g., RNA or DNA genes), host cell genome (e.g., to determine mutations), or cell free fetal DNA (cffDNA)), pathogens (e.g., viruses (e.g., HIV), parasites (e.g., P. falciparum), and/or bacteria (e.g., S. aureus)), metabolites (e.g., blood urea nitrogen, creatinine, bilirubin, carnosine, UDP-acetyl-glucosamine), hormones (e.g., thyroid, fertility/pregnancy, testosterone, cortisol), electrolytes (e.g., calcium, potassium, bicarbonate, chloride), lipids (e.g., HDL, LDL, VLDL, cholesterol, triglycerides), and/or small molecules (e.g., vitamins (e.g., folic acid, B vitamins, biotin) and/or sugars (e.g., glucose, Carbohydrate antigen 19-9 (sialyl-Lewis^{A}), sialyl-LewisX)).

In some embodiments, the method may be performed on any embodiment of the article, or combinations thereof, disclosed herein. In some embodiments, the article is configured to perform any embodiment of the method, or combinations thereof, disclosed herein.

In some, but not all, embodiments, the article and/or method has one or more advantages, such as short separation time, short collection time, ease of separation (e.g., without constant manual operation or the use of a centrifuge), ease of collection (e.g., without the use of centrifuges, such as high speed centrifuges, without the use of vacuum, and/or without the use of any additional instruments, such as pipettes), small surface area (e.g., small maximum horizontal dimension) of the article, ease of scaling up, ease of storage of the purified sample, large loading capacity, large volume recovery, low amounts of clogging of the article, low amounts of hemolysis in the recovered sample, high purity of the recovered sample, low amounts of mess (e.g., high containment of the blood within the article), low energy requirements, and/or ability to use whole blood samples without the need for dilution.

The following examples are intended to illustrate certain embodiments of the present invention, but do not exemplify the full scope of the invention.

### Example 1

This example studied the effect on volume of blood plasma recovered when varying the maximum horizontal dimension of the absorbent layer.

A separation device comprising a first layer and a second layer was used. The first layer and second layer each had a maximum horizontal dimension of 1.6 centimeters. The first layer comprised a treated polyester (Leukosorb). The second layer comprised a Pall Vivid plasma separation membrane (grade GR).

An absorbent layer comprising a blend of rayon and polypropylene (ShamWow) was used. Various maximum horizontal dimensions (i.e., 4 millimeters, 6 millimeters, 8 millimeters, 10 millimeters, and 16 millimeters) of the absorbent layer were tested while all other properties of the absorbent layer (e.g., thickness) were maintained.

The separation device was placed on top of the absorbent layer in a support structure. A 250-microliter sample of undiluted whole blood was placed on top of the separation device and 10 minutes were allowed for passive separation. After 10 minutes, the separation device was removed from the support structure, and a compression device was compressed against the absorbent layer, such that blood plasma from within the absorbent layer was transported from the absorbent layer to a capillary tube.

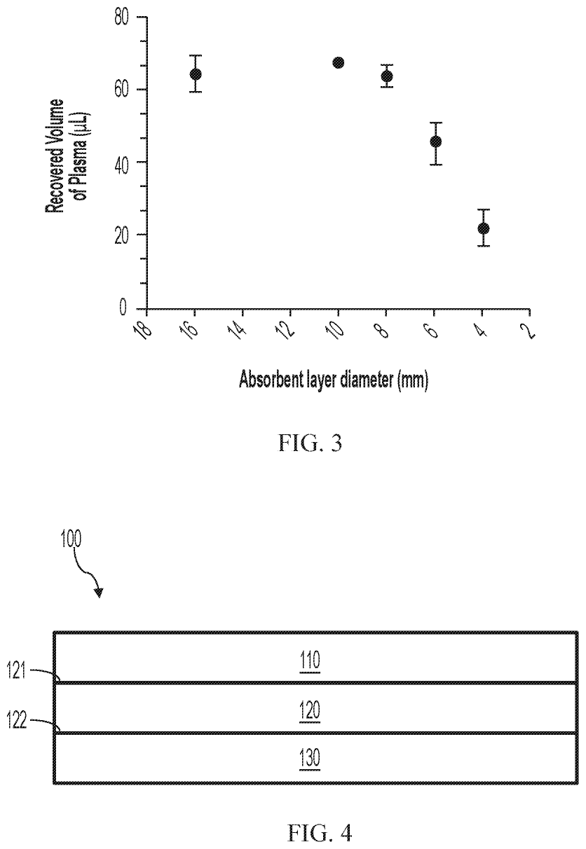

As shown in FIG. 3, the volume of blood plasma collected in the capillary tube was greater than or equal to 60 microliters (i.e., greater than or equal to 40% separation efficiency) when absorbent layers with maximum horizontal dimensions between 8 and 16 millimeters were used. The volume of blood plasma collected in the capillary tube was reduced (i.e., less than or equal to 50 microliters) when absorbent layers with maximum horizontal dimensions below 8 millimeters were tested. The data were collected in triplicate and the error bars in FIG. 3 represent the standard error of the mean. Accordingly, using an absorbent layer with a maximum horizontal dimension within a preferred range provided increased recovery and yield.

Hemolysis was evaluated visually and no evidence of hemolysis was detected for any of the absorbent layers tested. Accordingly, reducing the maximum horizontal dimension of the absorbent material did not increase the risk of hemolysis.

### Example 2

This example studied the effect on volume of blood plasma recovered when varying the maximum horizontal dimension of the first layer of the separation device.

Two separation devices comprising a first layer and a second layer were compared. In the first separation device, the first layer comprised a treated polyester (Leukosorb) and had a maximum horizontal dimension of 1.6 centimeters. The second layer comprised a Pall Vivid plasma separation membrane (grade GR) and had a maximum horizontal dimension of 1.6 centimeters. The second separation device was identical to the first except that the first layer had a maximum horizontal dimension of 1.3 centimeters.

An absorbent layer comprising a blend of rayon and polypropylene (ShamWow) and having a maximum horizontal dimension of 8 centimeters was used.

Either the first or second separation device was placed on top of the absorbent layer in a support structure. A 250-microliter sample of undiluted whole blood was placed on top of the separation device and 10 minutes were allowed for passive separation. After 10 minutes, the separation device was removed from the support structure, and a compression device was compressed against the absorbent layer, such that blood plasma from within the absorbent layer was transported from the absorbent layer to a capillary tube.

When the first separation device was used, 50.4±5.9 microliters of blood plasma were collected in the capillary tube (based on triplicate measurements). When the second separation device was used, 56.7±0.9 microliters of blood plasma were collected in the capillary tube (based on triplicate measurements). Accordingly, reducing the maximum horizontal dimension of the first layer provided increased recovery and yield in this example.

Hemolysis was evaluated visually and no evidence of hemolysis was detected for either separation device. Accordingly, reducing the maximum horizontal dimension of the first layer did not increase the risk of hemolysis in this example.

### Example 3

Described herein is, in accordance with some embodiments, an assembly of porous materials capable of obtaining high volumes (>60 μL) of pure plasma from whole blood using only passive methods in less than 10 minutes.

A pre-filter material was used to reduce the burden of excess blood cells from clogging the plasma separation membrane and minimize hemolysis independent of hematocrit. Separation and collection were facilitated by a super absorbent material in direct contact with the plasma separation membrane. The dual functionality of the collection pad permitted storage of purified plasma for shipping and future laboratory analysis similar to dried blood spot card technologies. The purity of collected plasma samples was evaluated by quantification of hemoglobin and the recovery of high and low concentration analytes of interest was evaluated.

### Experimental Design

### Device Design and Fabrication

The device comprised a pre-filter material, plasma separation membrane (PSM), and super absorbent material (FIG. 5).

The separation materials (e.g., pre-filter and plasma separation membrane) were affixed to the acrylic scaffold via rings of double-sided medical adhesive. The absorbent material was located in direct contact with the underside of the plasma separation membrane. Contact between each material was maintained by an acrylic scaffold and double-sided medical adhesive. The pre-filter material was designed to remove white blood cells from the sample matrix based on size exclusion and electrostatic interactions. The plasma separation membrane was designed to exclude all remaining white and red blood cells to produce pure plasma that can be simultaneously collected and stored by the underlying absorbent material.

All porous materials (e.g., pre-filter materials, PSM, and absorbent materials) were cut using a hammer-driven hole punch. Double-sided medical adhesive was patterned into rings using an automated knife plotter. Acrylic scaffolds were fabricated with a Trotech laser cutter.

The pore sizes of several materials evaluated for the device are shown in Table 1.

TABLE 1
Pore data for various materials.

| | | Surface area | Mode pore | Unique pore |
|---|---|---|---|---|
| Sample ID | Material | (m²/g) | diameter (μm) | diameters (μm) |
| TFN | Cellulose | 9.8 | 27.9 | 27.9, 19.8, 9.0 |
| Kapmat | Polyester | 17.1 | 159.4 | 159.4 |
| ShamWow | Rayon/Polypropylene | 32.2 | 91.6 | 91.6 |
| | Blend | | | |
| PSM-GR | Polysulfone | 43.7 | 16.3 | 23.9, 16.3, 10.0, |
| | | | | 7.4, 4.5, 0.15 |
| Leukosorb | Polyester | 43.7 | 19.5 | 19.5 |

### Operation of the Device

Plasma separation was initiated by applying a sample of whole blood to the top of the device and allowing it to sit for 5-10 minutes for separation to occur (see the schematic in FIG. 6). Purified plasma was collected by the absorbent material located beneath the plasma separation membrane. To terminate separation, the absorbent material was removed from the acrylic scaffold with a pair of tweezers and either (i) liquid plasma was recovered from the absorbent material via centrifugation, (ii) the porous material containing purified plasma was dried and stored for future laboratory analysis, or (iii) the absorbent material was immediately applied to a lateral flow test.

### Evaluation of Absorbency and Release for Porous Materials

The absorbency and release for the materials was determined as follows. The initial mass of each absorbent material was recorded (N=3, area=1 cm²). Then, each material was saturated in deionized water for 30 seconds and the saturated mass was recorded. The volume of water absorbed by each material was calculated using the density of water at ambient temperature. This value was normalized by the surface area of the material. This normalized value represented the “absorbency” of the material.

Then, the saturated absorbent materials were centrifuged to collect the water using a Swinex funnel attached to a 5-mL Eppendorf tube at an RCF of 800 g for 5 minutes. The Eppendorf tube was weighed empty and then with the released water, and the volume of water released by each material was calculated using the density of water at ambient temperature. This value represented the volume recovery. This volume was converted to a percentage of the water that was absorbed, and this value represented the “release” of the material.

### Quantification of the Recovered Plasma Volume and Calculation of Separation Efficiency

A centrifuge was used to quantify the volume of plasma collected in the devices as proof-of-concept (see, e.g., the schematic shown in FIG. 6). After plasma separation occurred, the absorbent material was removed from the acrylic scaffold using tweezers and added to a Swinex funnel attached to a 5-mL Eppendorf tube. The samples were centrifuged at an RCF of 800 g for 5 minutes to collect liquid plasma from the absorbent material. The mass of the liquid plasma was determined by calculating the difference between the initial mass of the 5-mL Eppendorf tube and the final mass after centrifugation. Then, the mass of the plasma sample was converted to recovered volume by using the average density of plasma (1.025 g/mL). The total theoretical plasma volume was determined based on the measured hematocrit value and input sample volume. Separation efficiency was defined as the ratio of collected plasma volume to total theoretical plasma volume.

### Recovery of Total Protein

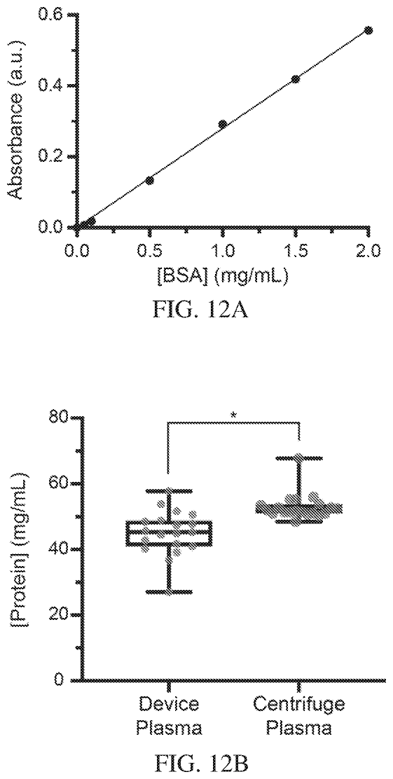

Recovery was calculated as the ratio of total protein in plasma samples obtained from the plasma separation device to the concentration of total protein in plasma samples obtained via centrifugation (see FIG. 12B). The Pierce 660 nm protein assay was used to quantify the total protein in plasma samples according to an established protocol. Briefly, 150 μL of the Pierce 660 reagent was added into a microwell plate, followed by 10 μL of diluted plasma (1:100 in 1×PBS). The microwell plate was incubated for 5 minutes at room temperature before reading at 660 nm using a Varioskan LUX microplate reader. A calibration curve was prepared using BSA solutions over a linear range from 0.05-2 mg/mL (as shown in FIG. 12A).

Recovery of High Abundance Protein h-IgG

Bio-Layer Inteferometry (K2 Octet, Pall Fortébio) was used to quantitate human immunoglobulin G (h-IgG) in reference plasma (i.e., obtained via centrifugation) and recovered plasma samples (i.e., obtained from the plasma separation device). A 96-well plate format with fiber-optic biosensors coated with Protein-A was used to measure the binding rate of h-IgG to Protein-A. Calibration curves were prepared using polyclonal h-IgG standards of known concentrations, ranging from 1-700 μg/mL (Pall Fortébio). The plasma samples were diluted 1:1000 in 1× Kinetics Buffer (Pall Fortébio) before quantitation to ensure the signal fell within the working range of the calibration curve.

The calibration curves were fit using a linear-point-to-point method, as described in the Protein-A Biosensor data sheet. The linear model was used to determine unknown concentrations for the reference plasma (N=20) and the recovered plasma (N=20). The two groups were statistically analyzed using a two-tailed Student's t-test with equal variances.

### Evaluation of Purity for Collected Plasma

The concentration of hemoglobin in recovered plasma was quantified to evaluate the purity of samples obtained by the plasma separation device. Extent of hemolysis was defined as the ratio of hemoglobin in plasma to total hemoglobin quantified according to an established method. For quantification of total hemoglobin in whole blood samples, a ratio of 1:250 was used (e.g., 4 μL of whole blood to 1 mL Drabkin's reagent containing 0.05% (v/v) Brij 25). Calibration curves were prepared daily using lyophilized hemoglobin standard rehydrated with diH2O (18 MΩ) and diluted over a range 3-20 g/dL. For quantification of hemoglobin in plasma samples, a ratio of 1:10 was used (e.g., 20 μL of whole blood to 0.2 mL Drabkin's reagent containing 0.05% (v/v) Brij 25). Calibration curves were prepared daily using lyophilized hemoglobin standard rehydrated with diH2O (18 MΩ) and diluted over a range 0.09-3 g/dL. The mixture was incubated at room temperature (i.e., 21° C.) for 15 minutes and absorbance was measured at 540 nm using a Varioskan LUX microplate reader. Plasma samples were collected from each plasma separation device and hemoglobin was quantified to determine extent of hemolysis against total hemoglobin concentration in whole blood. The LOD for both assays (i.e., 1:250 and 1:10 dilutions) were calculated using purified plasma obtained via centrifugation from three different donors.

### Tetanus Lateral Flow Test

Utility of the plasma separation device was demonstrated by applying collected plasma directly to commercially available lateral flow tests. All blood samples were collected from donors that had been vaccinated against tetanus and therefore contained tetanus antibodies. Positive controls from whole blood were prepared via centrifugation and negative controls from assay buffer provided with the lateral flow tests. 20 μL of plasma were applied to the sample input zone on the lateral flow test using a micropipette. 3 drops of assay buffer were then immediately added onto the sample input zone and 10 minutes were allowed to pass before scanning the results with an Epson V600 Perfection flatbed scanner at 800 DPI.

### RESULTS AND DISCUSSION

### Selection of Absorbent Materials (e.g., Third Layer)

A source of capillarity facilitated the performance of passive separation of plasma from whole blood. Capillarity was provided by the absorbent material, which was in direct contact with the separation materials above (FIG. 5). The desired material would provide (i) a fast wicking rate, (ii) high absorbency, and (iii) quantitative release of absorbed liquid. Three different wicking materials were tested: cellulose, polyester, and a rayon/polypropylene blend. The cellulose material had the lowest absorbency (65.0±7.0 μL/cm²) and released only 19% of the absorbed liquid (Table 2). In stark contrast, both the polyester and rayon/polypropylene blend materials absorbed 587.0±40.1 μL/cm² and 393.7±23.6 μL/cm², respectively. These super absorbent materials also yielded high percentages for the release of absorbed liquid at 93% (polyester) and 84% (rayon/polypropylene blend).

TABLE 2
Performance of various absorbent materials. Values represent the
average of five replicates and standard error of the mean.

| | Absorbency | Volume Recovery | |
|---|---|---|---|
| Material | (μL/cm² ) | (μL/cm² ) | Release |
| Cellulose | 65 ± 7 | 12 ± 7 | 19% |
| Polyester | 587 ± 40 | 544 ± 22 | 93% |
| Rayon/Polypropylene Blend | 393 ± 23 | 330 ± 27 | 84% |

Both the polyester and rayon/polypropylene blend materials in the device were evaluated for wicking ability in conjunction with the PSM. While the polyester material was more absorbent than the rayon/propylene blend, it caused more hemolysis of the blood sample. The rayon/polypropylene blend material did not cause hemolysis and therefore provided a better wicking source for separating plasma from whole blood in the device.

### Baseline Performance of the PSM (e.g., Second Layer)

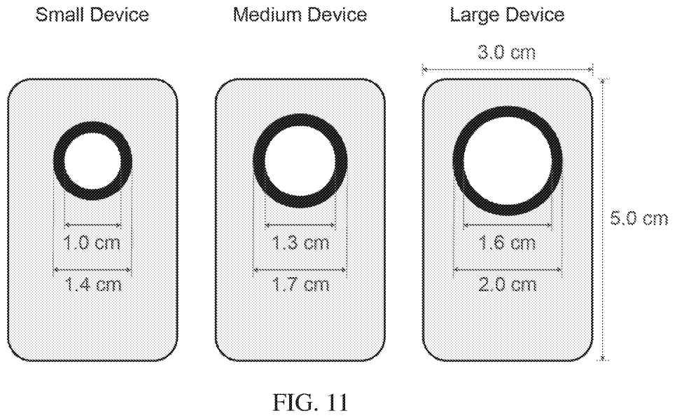

Three devices of different sizes (FIG. 11) were designed and tested with whole blood to establish baseline separation efficiencies using only a single layer of PSM. As shown in FIG. 11, the inner black ring on each device was the cavity ledge of acrylic (half depth cut, 0.317 cm), which provided physical support for the separation materials. The inner white circle was the open region of the device (full depth cut, 0.635 cm), which allowed direct contact between the absorbent material and the separation materials. The area of the plasma separation membrane determined the allowable sample input volume according to the manufacturer (40-50 μL cm-1). Theoretical sample input volumes were calculated for each device based on the minimum and maximum loading capacities for Vivid GR plasma separation membrane from Pall Corp (Table 3).

TABLE 3
Theoretical sample input volumes (μL)
for small, medium, and large devices.

| | Theoretical Sample Input Volume (μL) | | |
|---|---|---|---|
| Plasma Separation | Small Device | Medium Device | Large Device |
| Membrane Loading | (1.0 cm | (1.3 cm | (1.6 cm |
| Capacity (μL cm⁻¹) | diameter) | diameter) | diameter) |
| Minimum | 31.4 | 53.1 | 80.4 |
| Maximum | 39.3 | 66.3 | 100.5 |

A variety of PSMs were tested and the material with the greatest loading capacity (i.e., input volume of blood per area) and consistency was identified. Baseline separation for each device was measured after 10 minutes following sample addition and yielded a consistent volume range of 16-20% (maximum of 27.5 μL) of available plasma from a 250 μL sample input (Table 4). Minimal hemolysis was observed in all three devices (Table 4). The large device (1.6 cm diameter) achieved a higher degree of separation than the smaller devices under these conditions.

TABLE 4
Baseline data for each device with no pre-filter material. Sample input
volumes were 250 μL and 150 μL whole blood. Separation time (10 minutes) and
hematocrit (ca. 45%) were constant.

| | 250 μL | | | | 150 μL | | | |
|---|---|---|---|---|---|---|---|---|
| | Average Recovered | | Separation | Extent of | Average Recovered | | Separation | Extent of |
| Device | Volume (μL) | SEM | Efficiency | Hemolysis | Volume (μL) | SEM | Efficiency | Hemolysis |
| Small | 22.5 | 1.4 | 16.6% | 2.4% | 23.5 | 1.7 | 28.9% | 2.5% |
| (1.0 cm diameter) | | | | | | | | |
| Medium | 22.5 | 0.9 | 16.6% | 1.9% | 21.7 | 0.8 | 26.9% | 3.9% |
| (1.3 cm diameter) | | | | | | | | |
| Large | 27.1 | 1.9 | 20.0% | 2.7% | 22.3 | 2.1 | 27.6% | 2.5% |
| (1.6 cm diameter) | | | | | | | | |

The decreased separation of the smaller 250 μL devices (1.0 cm diameter) was attributed to an excess number of RBCs that clogged the pores of the PSM and impeded the flow of plasma through the membrane to the absorbent material below. In order to alleviate this burden on the PSM, a pre-filter material was included to remove RBCs and allow the plasma to flow through the membrane for collection. Potential pre-filter materials included fiberglass, polyester mesh with pore sizes ranging from 1-11 μm, and a fibrous membrane for the isolation of leukocytes from whole blood (Leukosorb, Pall Corp).

### Material Screen for Pre-filter (e.g., First Layer)

Fiberglass (Ahlstrom grade 8950) was initially selected for its propensity to act as a chromatographic material for blood separation without binding proteins or causing hemolysis. However, a single layer of fiberglass actually decreased the separation efficiency of the device by 3.5% (Table 6). The fiberglass was 0.25 mm thick with a reported void volume of 46 μL/cm². While fiberglass was capable of separating plasma from whole blood, the wicking rate and void volume of the material negatively impacted the performance of the device and required separation times in excess of 90 minutes.

The fibers of the polyester mesh did not absorb fluids or swell when in contact with liquid samples. This effectively lowered the void volume of the material, which increased the total recovery of plasma in the device. RBCs have an average size distribution of 6-8 μm and a biconcave disc geometry. However, since RBCs are easily deformable, a range of pore sizes were studied in an effort to create a pre-filter based on size exclusion for capturing RBCs. Initially, multiple layers of mesh with a pore size of 1 μm were tested as a pre-filter in a large plasma separation device (Table 5).

TABLE 5
Performance of multiple layers of mesh as pre-filter in
large acrylic devices. Sample input volume (150 μL
whole blood) and separation time (10 minutes) were constant.

| Large Acrylic | Average Recovered | | Separation | Extent of |
|---|---|---|---|---|
| Device | Volume (μL) | SEM | Efficiency | Hemolysis |
| Baseline | 22.3 | 2.1 | 27.6% | 2.5% |
| 1 layer | 30.0 | 2.0 | 36.8% | 1.3% |
| 2 layers | 28.6 | 1.5 | 35.1% | 3.2% |

In another embodiment, a mesh with a pore size of 11 μm was used to remove larger cells such as leukocytes (average diameter of 7-20 μm) from the sample matrix upon initiation of the device. The next layer had a pore size of 6 μm to remove any remaining leukocytes as well as a portion of RBCs. To ensure removal of all RBCs from the sample matrix prior to reaching the PSM, a final layer of polyester mesh with pore size of 1 μm was included. This construct of meshes acted as an effective pre-filter by increasing the separation efficiency by 9.6% and decreasing the extent of hemolysis by 1.2% within 10 minutes (Table 6). Iterations of this construct were investigated with single layers of polyester mesh (e.g., 1 μm, 6 μm, 11 μm), which yielded similar results. A maximum of 33.6% separation efficiency was achieved using two layers of polyester mesh with 1 and 6 μm pore sizes (Table 6).

TABLE 6
Performance of various pre-filter materials. Sample
input volume (250 μL whole blood), separation time
(10 minutes), and hematocrit (ca. 45%) were constant.

| | Average | | | |
|---|---|---|---|---|
| | Recovered | | Separation | Extent of |
| Pre-Filter Material | Volume (μL) | SEM | Efficiency | Hemolysis |
| Polyester Mesh (1 μm) | 37.3 | 0.7 | 27.5% | 1.7% |
| Polyester Mesh (6 μm) | 43.8 | 1.2 | 32.3% | 1.7% |
| Polyester Mesh (11 μm) | 45.3 | 0.6 | 33.4% | 1.5% |
| Polyester Mesh (1 + 6 μm) | 45.5 | 2.8 | 33.6% | 1.5% |
| Polyester Mesh (1 + 6 + | 40.1 | 1.8 | 29.6% | 1.5% |
| 11 μm) | | | | |
| Fiberglass | 22.4 | 5.0 | 16.5% | 3.8% |
| Leukosorb | 70.6 | 2.6 | 51.1% | 4.3% |

To complement the function of commercially available PSM for passively separating plasma from the complex matrix of whole blood, a fibrous membrane (Leukosorb by Pall Corp.) was used. Initial screening of this material yielded 51.1% separation efficiency and an average recovered volume of 70.6 μL of pure plasma (Table 6). Coupling the PSM and Leukosorb pre-filter allowed a high degree of separation of plasma from whole blood. To further characterize the performance of this device format, each parameter was optimized using the large device (1.6 cm diameter) to obtain the largest volume of plasma from the sample of whole blood.

Device Optimization using a Leukosorb First Layer and a PSM Second Layer

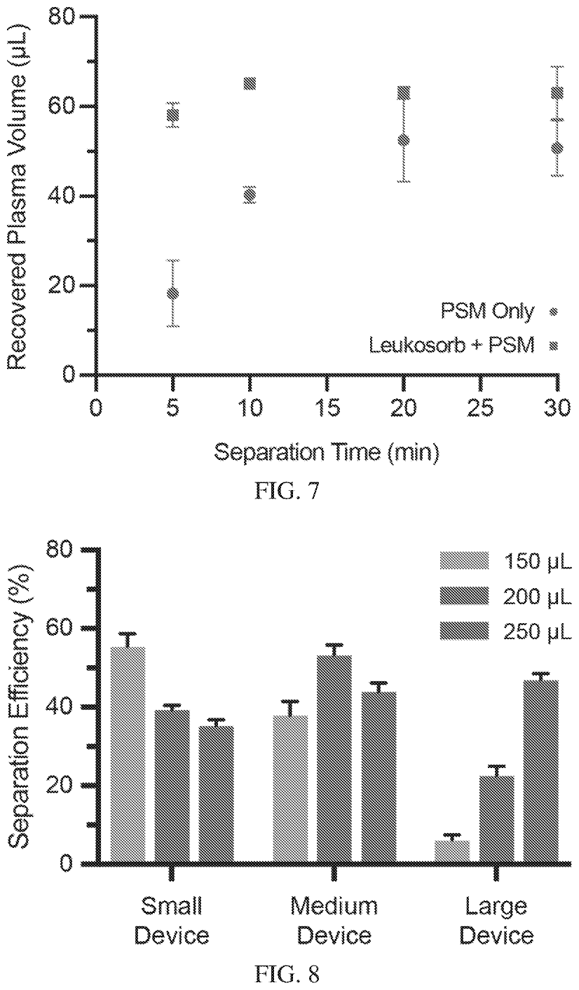

The combined theoretical void volume of the PSM (ca. 20 μL/cm²) and Leukosorb (ca. 40-70 μL/cm²) pre-filter with 1.6 cm diameter was 120-181 μL. The void volume was estimated to be approximately 150 μL by saturating the membranes with water and measuring the mass difference of the dry materials. While this was a considerable volume and directly impacted the maximum achievable separation efficiency, the addition of Leukosorb as a pre-filter increased the separation efficiency of the PSM three-fold after only 5 minutes of separation (FIG. 7).

The use of a Leukosorb prefilter with the PSM was evaluated for a 250 μL sample volume (47% Hct) in the small, medium, and large plasma separation device. The data was collected after 10 minutes were allowed for separation (Table 7).

TABLE 7
Data for small, medium, and large plasma separation device
with Leukosorb first layer and PSM second layer. Separation
time (10 min) and sample volume (250 μL) were constant.

| | Average Recovered | | Separation | Extent of |
|---|---|---|---|---|
| | Volume (μL) | SEM | Efficiency | Hemolysis |
| Large | 53.9 | 2.7 | 39.9% | 3.0% |
| Medium | 45.8 | 4.5 | 33.9% | 6.6% |
| Small | 29.7 | 1.7 | 21.9% | 4.1% |

Allowing separation to continue over a total of 30 minutes showed that Leukosorb and PSM together consistently outperformed PSM on its own. The maximum separation efficiency for PSM with no pre-filter was 35.6% after 20 minutes (Table 8). In contrast, PSM with a single layer of Leukosorb pre-filter yielded 43.5% separation efficiency after only 10 minutes (Table 9). Both device formats exhibited minimal hemolysis (≤2.4%) at the maximum separation efficiency.

TABLE 8
Data for a large plasma separation device (1.6 cm
diameter) with no pre-filter material (N = 3).

| Separation | Average Recovered | | Separation | Extent of |
|---|---|---|---|---|
| Time | Volume (μL) | SEM | Efficiency | Hemolysis |
| 5 min | 18.3 | 7.4 | 12.2% | 2.0% |
| 10 min | 40.3 | 1.8 | 26.8% | 2.4% |
| 20 min | 52.5 | 9.2 | 35.6% | 2.4% |
| 30 min | 50.7 | 6.2 | 34.3% | 1.5% |

TABLE 9
Data for large plasma separation device (1.6 cm diameter) with
a single layer of Leukosorb as the pre-filter material (N = 3).

| Separation | Average Recovered | | Separation | Extent of |
|---|---|---|---|---|
| Time | Volume (μL) | SEM | Efficiency | Hemolysis |
| 5 min | 58.1 | 2.6 | 38.8% | 0.8% |
| 10 min | 65.1 | 1.2 | 43.5% | 2.0% |
| 10 min | 72.1 | 2.0 | 48.5% | 3.8% |
| 20 min | 63.1 | 1.4 | 42.0% | 12.5% |
| 30 min | 63.1 | 5.9 | 42.0% | 2.1% |

Testing with Various Hematocrit Values

The number of RBCs in a sample of whole blood could affect both the total plasma yield as well as the plasma quality produced in separation. If the number of RBCs was increased, that could increase the burden on the PSM and result in unwanted hemolysis and sample contamination with intraerythrocytic contents. Therefore, the device was tested with samples of whole blood with varying hematocrit values (see Table 10 and Table 11). The maximum separation efficiency was 53.8% with an average recovered volume of 65.6 μL for a sample of whole blood with a hematocrit of 30% (see Table 11). Varying the hematocrit generally yielded similar values for recovered plasma volume, however, the separation efficiency generally decreased (see Table 11). Decreasing the hematocrit results in an increase in the theoretical volume of available plasma, which affects the value of separation efficiency. While an increase in average recovered volume (72.1 μL) for samples of whole blood was observed at 40% Hct, there was a decrease in average recovered volume (63.0 μL) for samples of whole blood at 35% Hct (Table 10).

TABLE 10
Plasma separation data for the large plasma separation device using
samples of whole blood over a range of hematocrit values (N = 5).

| | Average Recovered | | Separation | Extent of |
|---|---|---|---|---|
| Hematocrit | Volume (μL) | SEM | Efficiency | Hemolysis |
| 45% | 70.6 | 2.6 | 51.1% | 4.3% |
| d40% | 72.1 | 2.0 | 48.5% | 3.7% |
| 35% | 63.0 | 2.5 | 38.8% | 2.9% |

TABLE 11
Plasma separation data for the large plasma separation
device (1.6 cm diameter) using samples of whole blood
over a range of hematocrit values (20-60% Hct, N = 3).

| | Average Recovered | | Separation | Extent of |
|---|---|---|---|---|
| Hematocrit | Volume (μL) | SEM | Efficiency | Hemolysis |
| 20% | 60.6 | 3.3 | 30.1% | 1.9% |
| 30% | 65.6 | 3.9 | 53.8% | 1.0% |
| 40% | 60.4 | 4.8 | 40.6% | 0.9% |
| 50% | 60.0 | 2.5 | 48.0% | 0.9% |
| 60% | 43.9 | 0.8 | 44.7% | 1.1% |

To optimize the separation efficiency achieved by the plasma separation device, each device (small, medium, large) was tested with a range of input sample volumes from 150-250 μL at a constant hematocrit value of 45% (FIG. 8). Each device had a specific input volume that resulted in maximum separation efficiency after 10 minutes of separation with a constant hematocrit (45% Hct). The small device (1.0 cm diameter) produced optimal separation efficiency of 55.5% with a sample input of 150 μL. The medium device (1.3 cm diameter) produced optimal separation efficiency of 53.3% with a sample input of 200 μL. The large device (1.6 cm diameter) produced optimal separation efficiency of 47.0% with a sample input of 250 μL. The corresponding average recovered volume of plasma can be found in Table 12 for each device. Each device consistently showed a decrease in separation efficiency when the input sample volume deviated from the optimal input sample volume.

TABLE 12
Data for plasma separation devices (small, medium, and large)
with a single layer of Leukosorb as the pre-filter (N = 5).

| | | Average | | | |
|---|---|---|---|---|---|
| Sample Input | Device | Recovered | | Separation | Extent of |
| Volume (μL) | Size | Volume (μL) | SEM | Efficiency | Hemolysis |
| 150 | Small | 45.3 | 2.6 | 55.5% | 5.3% |
| | Medium | 30.7 | 2.7 | 38.0% | 0.7% |
| | Large | 5.1 | 1.1 | 6.2% | 0.6% |
| 200 | Small | 42.8 | 1.2 | 39.4% | 4.0% |
| | Medium | 57.3 | 2.7 | 53.3% | 5.4% |
| | Large | 24.8 | 2.5 | 22.6% | 0.6% |
| 250 | Small | 47.8 | 1.8 | 35.4% | 9.4% |
| | Medium | 59.1 | 2.8 | 44.1% | 2.2% |
| | Large | 64.8 | 2.2 | 47.0% | 4.2% |

### Plasma Quality

Pure plasma obtained from standard methods-such as centrifugation-contains various proteins, solutes, and platelets. These include analytes of interest which must be conserved during separation so that the sample is relevant for subsequent analysis and diagnostic utility. Plasma sample impurity may arise from ruptured red blood cells and the release of intraerythrocytic analytes such as hemoglobin. The quality of plasma obtained from the device was evaluated by quantifying (i) total protein, (ii) specific h-IgG (high abundance), and (iii) specific IL-X (low abundance). Purity was measured by quantification of hemoglobin and diagnostic utility was demonstrated by direct application of collected plasma to a commercially available lateral flow test for the tetanus antibody. Whole blood from a single donor was applied to 20 plasma separation devices and a reference sample of pure plasma was prepared via centrifugation.

In order to improve the elution process, data evaluating the protein recovery was collected over varying buffer compositions (Table 13). Once the samples were fully dried, a volume of buffer (containing various surfactants) was added to the absorbent material to rehydrate the analytes found in plasma. Then the sample was extracted from the absorbent material via the same centrifugation method previously described.

TABLE 13
Total protein recovery as a result of varying buffer compositions

| | [Protein] | SEM | Protein |
|---|---|---|---|
| Eluent | (g/L) | (g/L) | Recovery |
| Water | 34.8 | 9.1 | 61% |
| Phosphate Buffered Saline | 44.3 | 0.5 | 91% |
| PBS + Surfactant 1 | 46.5 | 0.8 | 97% |
| PBS + Surfactant 2 | 46.8 | 0.9 | 97% |
| PBS + Surfactant 3 | 46.5 | 0.6 | 97% |
| PBS + Surfactant 4 | 47.1 | 1.0 | 98% |
| PBS + Surfactant 5 | 47.0 | 0.6 | 98% |
| PBS + Surfactant 6 | 48.3 | 0.0 | 101% |

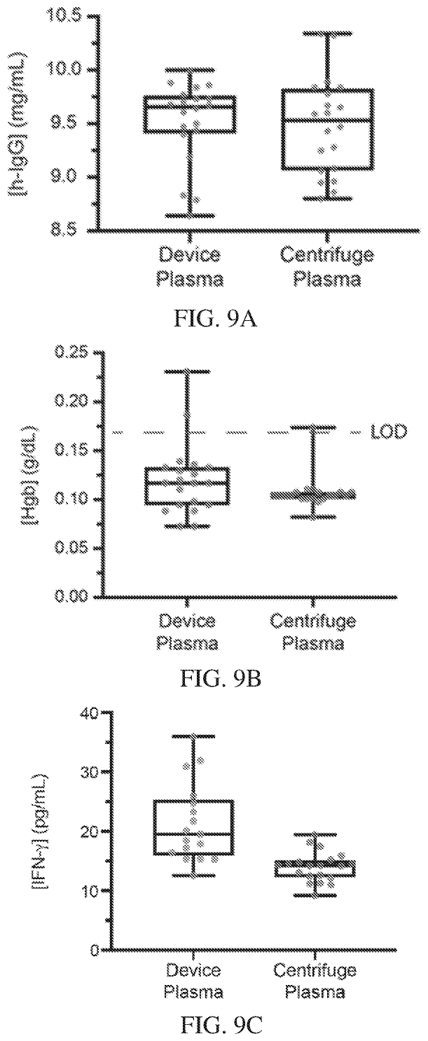

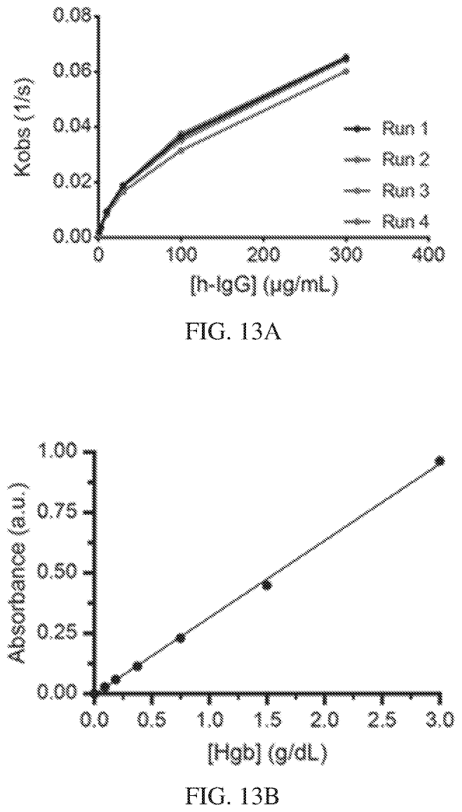

Total protein analysis of plasma obtained from the device yielded a recovery of 86.2% using the Pierce 660 assay (FIGS. 13A-13B). A two-tailed Student's t-test yielded a p-value of <0.0001, indicating a loss of total protein between the two sets of plasma samples. Adsorption of proteins (e.g., albumins and globulins) was expected in porous materials. To better evaluate the loss of proteins in the device, specific proteins of interest were quantified at high (h-IgG) and low (h-IFNγ) quantities. The concentration of h-IgG in recovered plasma from the device was nearly identical to the concentration in reference plasma, suggesting that there was no apparent loss of human IgG to the materials in the plasma separation device (FIG. 9A and Table 14).

TABLE 14
Human IgG concentration in reference and recovered plasma as
quantified by Bio-Layer Interferometry. The average concentrations
and standard deviations are nearly identical between the reference
plasma (N = 20) and the recovered plasma (N = 20).

| | Centrifuge Plasma | Device Plasma |
|---|---|---|
| Average [h-IgG] (mg/mL) | 9.48 | 9.52 |
| SD | 0.45 | 0.38 |

A two-tailed Student's t-test yielded a p-value of 0.786, providing no evidence of a difference in h-IgG concentration between the two sets of plasma samples (FIG. 9A).

Purity of the plasma collected with the plasma separation device was verified by quantification of released hemoglobin as a function of hemolysis (FIGS. 13A-13B). The LOD was calculated as 0.17 g/dL hemoglobin using purified plasma (i.e., obtained via centrifugation) from three different donors (FIG. 13B). Both the reference and recovered samples yielded hemoglobin concentrations below the LOD at 0.11±0.02 and 0.12±0.04 g/dL, respectively (FIG. 9B). Low concentrations of released hemoglobin indicated a lack of hemolysis and subsequent high purity of plasma samples obtained from the device.

The amount of the low concentration analyte (pg/mL), IFN-γ, present in the recovered plasma sample was in agreement with that in the reference plasma sample, as shown in FIG. 9C. Quantitation of IFN-γ by qPCR using a ProQuantum immunoassay kit showed no loss of IFN-γ in the recovered plasma sample even at extremely low concentrations, indicating that the quality of the plasma is conserved even for low abundance proteins. A two-tailed Student's t-test yielded a p-value of <0.001 and the difference in average concentrations of IFN-γ between the recovered plasma sample and the reference plasma sample was 7.3 pg/mL, which is within the tolerance of the ProQuantum immunoassay kit.

Additionally, the recovery of HIV RNA in the recovered plasma sample was also evaluated. Simulated samples of HIV-positive whole blood at a viral load of 50,000 copies/mL were prepared by spiking plasma from an HIV-positive patient into whole blood from an HIV-negative patient. RT-qPCR was used to detect and quantify the presence of HIV RNA. All experiments were performed in triplicate. The plasma recovered from the simulated HIV-positive whole blood samples had an average threshold cycle value (Ct, unitless) of 23.3±0.6, while the average Ct for control plasma samples, obtained from the simulated whole blood via centrifugation, was 22.1±0.3. These Ct values correlate to 43.3% elution efficiency for total HIV RNA collected from the recovered plasma sample.

HIV-positive plasma was tested on the device as a less complex sample matrix than whole blood. When HIV-positive plasma was added to the device, a very slight difference in Ct values (24.1 vs 24.8) was observed. The loss of efficiency with whole blood samples was likely due to matrix effects, where some HIV virions were nonspecifically filtered during the plasma separation process due to interactions with the cells contained in the otherwise naïve blood.

### Demonstration of a Diagnostic Test

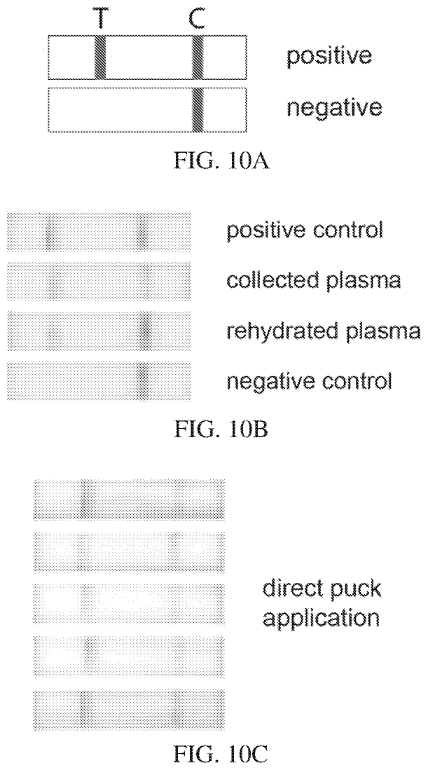

The goal was to produce a device capable of passive plasma separation for use at the point-of-care in resource limited settings. While the majority of these analyses were performed on liquid plasma samples collected via centrifugation following separation in the device, this example also demonstrated the direct utility of the device for performing a lateral flow test without centrifugation (FIG. 7). Samples collected with the plasma separation device were (i) recovered in liquid form using a centrifuge (FIG. 10B), (ii) dried overnight in the absorbent puck, rehydrated with elution buffer, recovered in liquid form using a centrifuge (FIG. 10B), and (iii) directly applied to a lateral flow test without centrifugation (FIG. 10C) (e.g., like a stamp). In agreement with the positive samples obtained by centrifugation (FIG. 10A), positive results were obtained for every condition tested using undiluted human plasma obtained from the device. Slight attenuation of the control line occurred when the sample was directly applied from the absorbent puck (FIG. 10C), however, the diagnostic output of the lateral flow test was unaffected.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A kit, comprising:
a support structure comprising an inlet, an outlet, and a channel between the inlet and the outlet;
a separation device;
an absorbent layer; and
a compression device;
wherein the absorbent layer and support structure are configured such that the absorbent layer can be positioned in the support structure and be in fluidic connection with the inlet and the outlet of the support structure;
wherein the separation device is removable from the support structure;
wherein the outlet is a vessel and/or is configured to be in fluidic connection with a vessel; and
wherein the compression device and support structure are configured such that at least a portion of the compression device can be positioned at the inlet.

2. A kit, comprising:
a support structure comprising an inlet, an outlet, and a channel between the inlet and the outlet; and
an absorbent layer;
wherein the absorbent layer and support structure are configured such that the absorbent layer can be positioned in the support structure and be in fluidic connection with the inlet and the outlet of the support structure;
wherein the outlet is a vessel and/or is configured to be in fluidic connection with a vessel; and
wherein the absorbent layer has an absorbency of greater than or equal to 80 microliters/cm² and less than or equal to 600 microliters/cm².

3. A kit, comprising:
a support structure comprising an inlet, an outlet, and a channel between the inlet and the outlet;
a separation device, wherein the separation device is removable from the support structure, and wherein the separation device comprises a first layer and a second layer; and
an absorbent layer;
wherein the absorbent layer and support structure are configured such that the absorbent layer can be positioned in the support structure and be in fluidic connection with the inlet and the outlet of the support structure; and
wherein the outlet is a vessel and/or is configured to be in fluidic connection with a vessel.

4. A kit, comprising:
a support structure comprising an inlet, an outlet, and a channel connecting the inlet and the outlet; and
an absorbent layer;
wherein the absorbent layer and support structure are configured such that the absorbent layer can be positioned in the support structure and be in fluidic connection with the inlet and the outlet of the support structure;
wherein the outlet is a vessel and/or is configured to be in fluidic connection with a vessel; and
wherein the channel of the support structure has an internal volume of less than or equal to 10 milliliters.

5. A method, comprising:
in a support structure comprising an inlet, an outlet, a channel between the inlet and the outlet, a separation device positioned in the support structure, and an absorbent layer positioned in the support structure, performing the steps of:
passing a blood sample across the separation device to the absorbent layer, such that a blood sample with reduced number of red blood cells is collected inside the absorbent layer;
removing the separation device from the support structure after the blood sample with reduced number of red blood cells has been passed into the absorbent layer; and
compressing a compression device against the absorbent layer after the separation device has been removed from the support structure.

6. The kit of any one of claim 2 or 4, wherein the kit further comprises a separation device, and wherein the separation device is removable from the support structure.

7. The kit of any one of claims 2-4, wherein the kit further comprises a compression device.

8. The kit of claim 7, wherein the compression device and support structure are configured such that at least a portion of the compression device can be positioned at the inlet.

9. The kit of any preceding claim, wherein the separation device is positioned in the support structure.

10. The kit of any preceding claim, wherein the absorbent layer is positioned in the support structure.

11. A method, comprising:
using the kit of any preceding claim, performing the step of:
passing a blood sample across the separation device to the absorbent layer, such that a blood sample with reduced number of red blood cells is collected inside the absorbent layer.

12. The method of claim 11, further comprising removing the separation device from the support structure after the blood sample with reduced number of red blood cells has been passed into the absorbent layer.

13. The method of claim 12, further comprising compressing a compression device against the absorbent layer after the separation device has been removed from the support structure.

14. The method of any one of claims 11-13, further comprising collecting the blood sample with reduced number of red blood cells in a vessel.

15. The method of any preceding claim, further comprising collecting the blood sample with reduced number of red blood cells in a vessel after compressing the compression device against the absorbent layer.

16. The kit or method of any preceding claim, wherein the absorbent layer has an absorbency of greater than or equal to 80 microliters/cm² and less than or equal to 600 microliters/cm².

17. The kit or method of any preceding claim, wherein the separation device comprises a first layer and a second layer.

18. The kit or method of any preceding claim, wherein the channel of the support structure has an internal volume of less than or equal to 10 milliliters.

19. The kit or method of any preceding claim, wherein the compression device is removable from the support structure.

20. The kit or method of any preceding claim, wherein the compression device is not removable from the support structure after it is compressed against the absorbent layer.

21. The kit or method of any preceding claim, wherein the absorbent layer is positioned in the channel of the support structure.

22. The kit or method of any preceding claim, wherein the absorbent layer is secured to the support structure using adhesive.

23. The kit or method of any preceding claim, wherein the absorbent layer is secured to the support structure due to its positioning between ridges in the support structure.

24. The kit or method of any preceding claim, wherein the compression device comprises a cap.

25. The kit or method of claim 24, wherein the cap is configured to seal the inlet of the support structure such that liquid cannot be transported from the absorbent layer through the inlet to an exterior of the support structure and/or liquid cannot be transported from an exterior of the support structure through the inlet to the absorbent layer.

26. The kit or method of any preceding claim, wherein the compression device comprises a plunger that is configured to compress the absorbent layer when the compression device is placed at the inlet of the support structure.

27. The kit or method of any preceding claim, wherein the inlet and/or channel of the support structure comprises an interior surface and an exterior surface, wherein the exterior surface comprises one or more ridges.

28. The kit or method of claim 27, wherein the one or more ridges of the exterior surface of the inlet and/or channel are configured to secure the compression device to the support structure.

29. The kit or method of any preceding claim, wherein the compression device is configured to screw onto the support structure.

30. The kit or method of any one of claims 27-29, wherein the compression device is configured to screw onto the support structure via the one or more ridges of the exterior surface of the inlet and/or channel.

31. The kit or method of any preceding claim, wherein the support structure and/or the compression device are 3D printed.

32. The kit or method of any preceding claim, wherein the vessel comprises a capillary tube.

33. The kit or method of any preceding claim, wherein the outlet is the vessel.

34. The kit or method of any preceding claim, wherein the outlet is configured to be in fluidic connection with the vessel.

35. The kit or method of any preceding claim, wherein the kit is configured to passively separate a blood sample to produce a blood sample with reduced number of red blood cells and collect the blood sample with reduced number of red blood cells and/or the method comprises passively separating a blood sample to produce a blood sample with reduced number of red blood cells and collecting the blood sample with reduced number of red blood cells.

36. The kit or method of any preceding claim, wherein the absorbent layer is porous.

37. The kit or method of any preceding claim, wherein the absorbent layer has an absorbency of greater than or equal to 200 microliters/cm² and less than or equal to equal to 450 microliters/cm².

38. The kit or method of any preceding claim, wherein the absorbent layer is configured to absorb blood plasma.

39. The kit or method of any preceding claim, wherein the absorbent layer has a mode pore size of greater than or equal to 20 microns and less than or equal to 150 microns.

40. The kit or method of any preceding claim, wherein the absorbent layer has a mode pore size of greater than or equal to 75 microns and less than or equal to 125 microns.

41. The kit or method of any preceding claim, wherein the absorbent layer has a release of greater than or equal to 35%.

42. The kit or method of any preceding claim, wherein the absorbent layer has a release of greater than or equal to 50%.

43. The kit or method of any preceding claim, wherein the absorbent layer has a release of greater than or equal to 70%.

44. The kit or method of any preceding claim, wherein the absorbent layer comprises rayon and/or polyester.

45. The kit or method of any preceding claim, wherein the absorbent layer comprises a blend of rayon and polypropylene.

46. The kit or method of any preceding claim, wherein the absorbent layer has a thickness of greater than or equal to 200 microns and less than or equal to 800 microns.

47. The kit or method of any preceding claim, wherein the absorbent layer has a thickness of greater than or equal to 250 microns and less than or equal to 500 microns.

48. The kit or method of any preceding claim, wherein the first layer is porous and has a first mode pore size that is greater than or equal to 1 micron and less than or equal to 30 microns.

49. The kit or method of any preceding claim, wherein the second layer is porous and greater than or equal to 20% of the pores of the second layer have a pore size of less than or equal to 20 microns.

50. The kit or method of any preceding claim, wherein the second layer is positioned between the first layer and the absorbent layer.

51. The kit or method of any preceding claim, wherein the first layer is in direct contact with the second layer and/or the second layer is in direct contact with the absorbent layer.

52. The kit or method of any one of claims 48-51, wherein the first mode pore size is greater than or equal to 2 microns and less than or equal to 25 microns.

53. The kit or method of any one of claims 48-52, wherein the first mode pore size is greater than or equal to 15 microns and less than or equal to 25 microns.

54. The kit or method of any preceding claim, wherein the second layer has a second mode pore size, and the second mode pore size is greater than or equal to 2 microns and less than or equal to 30 microns.

55. The kit or method of claim 54, wherein the second mode pore size is greater than or equal to 10 microns and less than or equal to 20 microns.

56. The kit or method of any preceding claims, wherein the second layer has a second mode pore size, and the second mode pore size is smaller than the first mode pore size of the first layer.

57. The kit or method of any preceding claim, wherein greater than or equal to 50% of the pores of the second layer have a pore size of less than or equal to 20 microns.

58. The kit or method of any preceding claim, wherein greater than or equal to 20% of the pores of the second layer have a pore size of less than or equal to 10 microns.

59. The kit or method of any preceding claim, wherein the second layer has a first surface and a second surface, wherein the second layer has a gradient in mode pore size between the first surface and the second surface, such that the first surface, which faces the first layer, has a mode pore size, the second surface, which faces the absorbent layer, has a mode pore size, and the mode pore size of the second surface is smaller than the mode pore size of the first surface.

60. The kit or method of claim 59, wherein a ratio of the mode pore size of the first surface to the mode pore size of the second surface is greater than or equal to 5:1 and less than or equal to 1,000:1.

61. The kit or method of any one of claims 59-60, wherein a ratio of the mode pore size of the first surface to the mode pore size of the second surface is greater than or equal to 100:1 and less than or equal to 200:1.

62. The kit or method of any preceding claim, wherein there are no intervening layers between the first layer and the second layer and/or the second layer and the absorbent layer.

63. The kit or method of any preceding claim, wherein the first layer is adhered to the second layer.

64. The kit or method of any preceding claim, wherein the first layer is adhered to the second layer with adhesive around the perimeter of the first layer and/or second layer where they are in contact, and wherein the adhesive creates a full seal around the perimeter.

65. The kit or method of any preceding claim, wherein the adhesive has a thickness of greater than or equal to 0.03 millimeters and less than or equal to 0.2 millimeters.

66. The kit or method of any preceding claim, wherein the adhesive comprises a UV cured adhesive.

67. The kit or method of any preceding claim, wherein the first layer has a thickness of greater than or equal to 150 microns and less than or equal to 500 microns.

68. The kit or method of any preceding claim, wherein a maximum horizontal dimension of the first layer, the second layer, and/or the absorbent layer is greater than or equal to 4 millimeters and less than or equal to 500 millimeters.

69. The kit or method of any preceding claim, wherein a maximum horizontal dimension of the first layer and/or the second layer is greater than or equal to 10 millimeters and less than or equal to 20 millimeters.

70. The kit or method of any preceding claim, wherein a maximum horizontal dimension of the absorbent layer is greater than or equal to 4 millimeters and less than or equal to 16 millimeters.

71. The kit or method of any preceding claim, wherein a maximum horizontal dimension of the absorbent layer is greater than or equal to 7 millimeters and less than or equal to 9 millimeters.

72. The kit or method of any preceding claim, wherein the first layer comprises greater than or equal to 2 sub-layers and less than or equal to 4 sub-layers.

73. The kit or method of claim 72, wherein each of the sub-layers has a different mode pore size and the sub-layers are arranged such that a gradient in mode pore size is formed.

74. The kit or method of any preceding claim, wherein the first layer, second layer, and absorbent layer are stacked vertically in the support structure.

75. The kit or method of any preceding claim, wherein the first layer comprises fiberglass, polyester, a fibrous membrane, polyether sulfone, polyester, nylon, and/or mesh.

76. The kit or method of any preceding claim, wherein the first layer comprises polyester.

77. The kit or method of any preceding claim, wherein the second layer comprises a polymer.

78. The kit or method of any preceding claim, wherein the second layer comprises polyether sulfone.

79. The kit or method of any preceding claim, wherein the second layer comprises a plasma separation membrane.

80. The kit or method of any preceding claim, wherein the support structure comprises a plastic, an acrylic, and/or a metal.

81. The kit or method of any preceding claim, wherein the first layer has an absorbency of greater than or equal to 10 microliters/cm² and less than or equal to 100 microliters/cm².

82. The kit or method of any preceding claim, wherein the first layer has an absorbency of greater than or equal to 20 microliters/cm² and less than or equal to 50 microliters/cm².

83. The kit or method of any preceding claim, wherein the second layer has an absorbency of greater than or equal to 10 microliters/cm² and less than or equal to 50 microliters/cm².

84. The kit or method of any preceding claim, wherein the second layer has an absorbency of greater than or equal to 15 microliters/cm² and less than or equal to 25 microliters/cm².

85. The method of any preceding claim, wherein passing the blood sample across the separation device to produce a blood sample with reduced number of red blood cells and/or passing the blood sample with reduced number of red blood cells into the absorbent layer is passive.

86. The kit of method of any preceding claim, wherein the blood sample is undiluted whole blood.

87. The kit or method of any preceding claim, wherein passing the blood sample across the separation device to produce a blood sample with reduced number of red blood cells and passing the blood sample with reduced number of red blood cells into the absorbent layer is accomplished within less than or equal to 30 minutes.

88. The kit or method of any preceding claim, wherein passing the blood sample across the separation device to produce a blood sample with reduced number of red blood cells and passing the blood sample with reduced number of red blood cells into the absorbent layer is accomplished within less than or equal to 10 minutes.

89. The kit or method of any preceding claim, wherein passing the blood sample across the separation device to produce a blood sample with reduced number of red blood cells and passing the blood sample with reduced number of red blood cells into the absorbent layer is accomplished within less than or equal to 5 minutes.

90. The kit or method of any preceding claim, wherein the blood sample has a volume of greater than or equal to 25 microliters.

91. The kit or method of any preceding claim, wherein the blood sample has a volume of greater than or equal to 100 microliters.

92. The kit or method of any preceding claim, wherein the blood sample has a volume of less than or equal to 500 microliters.

93. The method of any preceding claim, wherein collecting the blood sample with reduced number of red blood cells in the vessel is accomplished without the use of a centrifuge.

94. The kit or method of any preceding claim, wherein the separation device has a separation efficiency of greater than or equal to 10%.

95. The kit or method of any preceding claim, wherein the separation device has a separation efficiency of greater than or equal to 30%.

96. The kit or method of any preceding claim, wherein the separation device has a separation efficiency of greater than or equal to 50%.

97. The kit or method of any preceding claim, wherein the blood sample with reduced number of red blood cells has less than or equal to 15% hemolysis.

98. The kit or method of any preceding claim, wherein the blood sample with reduced number of red blood cells has less than or equal to 7% hemolysis.

99. The method of any preceding claim, wherein the method comprises using the blood sample in a clinical test, diagnostic health test, clinical chemistry assay, immunoassay, immunochromatographic assay for antibodies, quantification of cytokines, amplification of viral RNA, rapid dipstick test, HIV viral load assay, cholesterol test, metabolite panel, serology for an infectious disease, and/or therapeutic drug monitoring.

100. The kit or method of any preceding claim, wherein the blood sample with reduced number of red blood cells has less than or equal to 5% of the red blood cells in the blood sample.

101. The kit or method of any preceding claim, wherein the blood sample with reduced number of red blood cells has less than or equal to 2% of the red blood cells in the blood sample.

102. The kit or method of any preceding claim, wherein the volume of the blood sample with reduced number of red blood cells passed into the absorbent layer and/or collected in the vessel is greater than or equal to 20 microliters and less than or equal to 150 microliters.

103. The kit or method of any preceding claim, wherein the volume of the blood sample with reduced number of red blood cells passed into the absorbent layer and/or collected in the vessel is greater than or equal to 30 microliters and less than or equal to 150 microliters.

104. The kit or method of any preceding claim, wherein the volume of the blood sample with reduced number of red blood cells passed into the absorbent layer and/or collected in the vessel is greater than or equal to 50 microliters and less than or equal to 150 microliters.

105. The kit or method of any preceding claim, wherein the blood sample with reduced number of red blood cells has a level of an analyte of interest that is greater than or equal to 40% and less than or equal to 100% a level of the analyte of interest in the blood sample.

106. The kit or method of any preceding claim, wherein the blood sample with reduced number of red blood cells has a level of an analyte of interest that is greater than or equal to 80% and less than or equal to 100% a level of the analyte of interest in the blood sample.

107. The kit or method of any one of claims 105-106, wherein the analyte of interest comprises proteins, nucleic acids, pathogens, metabolites, hormones, electrolytes, lipids, and/or small molecules.

108. The kit or method of any preceding claim, wherein the vessel has an internal volume of less than or equal to 10 milliliters.

109. The kit of any preceding claim, wherein the kit comprises an additional absorbent layer with a different maximum horizontal dimension than the absorbent layer and/or the kit comprises an additional separation device with a different maximum horizontal dimension than the separation device.

110. The kit of any preceding claim, wherein the support structure is configured to be used with separation devices of different sizes and/or absorbent layers of different sizes.

111. The method of any preceding claim, further comprising selecting a maximum horizontal dimension of an absorbent layer and/or a maximum horizontal dimension of a separation device based on a desired volume of blood sample and/or a desired volume of blood sample with reduced number of red blood cells passed into the absorbent layer and/or collected in the vessel.
